(19) United States
(12) Patent Application Publication
(10) Pub. No.: US 2010/0310548 A1
(43) Pub. Date: Dec. 9, 2010

(54) Methods of Manufacturing a Biologic Using a Stable Storage Intermediate

(75) Inventors: Ping Yeh, Solana Beach, CA (US); Jorg Thommes, San Diego, CA (US); Jorg Thommes, San Diego, CA (US); Wolfgang Noe, San Diego, CA (US); Wolfgang Berthold, Solana Beach, CA (US)

Correspondence Address: STERNE, KESSLER, GOLDSTEIN & FOX, P.L.L.C., 1100 NEW YORK AVE., N.W., WASHINGTON, DC, 20005, US

(73) Assignee: Biogen Idec MA Inc., Cambridge, MA (US)

(21) Appl. No.: 12/738,091

(22) PCT Filed: Oct. 15, 2008
(86) PCT No.: PCT/US08/11764

§ 371 Date: Aug. 26, 2010

Related U.S. Application Data
(60) Provisional application No. 60/960,789, filed on Oct. 15, 2007.

(51) Int. Cl. A61K 39/395; C12P 1/00; C12P 21/00; A61P 37/00

(52) U.S. Cl. 424/130.1; 435/41; 435/71.1

(57) ABSTRACT

The present invention is directed to methods of isolating and purifying protein from a bioreactor process. The invention relates to employing a protein phase separation methodology to isolate or purify protein product in the forms of solid, semi-solid, or suspension during a protein manufacturing purification process as a stable storage intermediate. This approach is designed to allow the isolated protein product (purified or partially purified) to be stored over an extended period of time prior to further protein purification steps.

### BACKGROUND OF THE INVENTION

For a number of years, many therapeutic agents have been chemically synthesized small molecules. However, recent advances in biological chemistry, genetics and molecular biology have led to more frequent identification of potential protein-based drugs. These include, for example, cytokines, hormones, clotting factors, growth factors, antibodies and antigenic peptides for vaccines. In addition, some of the newer protein therapeutics such as antibodies and Fc-fusion proteins require higher doses and may be used to treat larger patient populations than some of the earlier protein therapeutics such as vaccines and growth factors. Shukla et al. Journal of Chromatography B 848: 28-39 (2007). These changes have resulted in an increased demand for protein therapeutics. However, it is not technically feasible to simply scale up routine laboratory techniques for protein production and purification in order to meet the increased demand. Id. Thus, there is currently a need for improving industrial-scale protein production and purification processes.

The manufacture of protein pharmaceuticals can be thought of as a multi-step process. The first step is bulk drug substance (BDS) manufacturing and typically involves large scale protein production followed by protein purification. The BDS generally must meet particular release specifications that include quality attributes, strength, purity, identity and safety assessments. The next step is drug product (DP) manufacturing which is the conversion of the bulk drug substance into a final pharmaceutical formulation that can be packaged and delivered to doctors and patients for therapeutic use. DP manufacturing steps are sometimes referred to as “fill-finish” operations.

The demand for more protein pharmaceuticals has led to a number of innovations in each of the individual steps of the process. However, innovations that increase the efficiency of one step of the process often require an equivalent increase in efficiency of each of the downstream steps in order to prevent bottlenecks in the overall process. For example, the step of protein production is frequently carried out by growing a large number of recombinant cells in a bioreactor, and advances over the past several years have increased the capacity of bioreactors up to as much as 20,000 liters. Kelley, B. Biotechnol.Prog. 23: 995-1008 (2007). In order for these advances to increase the overall efficacy of the manufacturing process though, they must be matched by increases in the efficacy of the downstream purification steps or else there will be a bottleneck between protein production and protein purification. Methods to improve purification are underway and some predict that the efficacy of purification processes will soon match the current scale of protein production. See id. However, increasing the efficiency of only selected purification steps may simply lead to a bottleneck further downstream in the process.

Typically, manufacturing processes for biologic therapeutics are carried out in an uninterrupted mode from the start of the bioreactor train and throughout purification to formation of the BDS. The BDS stage frequently represents the first true hold point in this chain of activities. These uninterrupted operations contribute to the creation of the bottlenecks mentioned above. By executing a continuous flow of operations, efficiency gains in one operation that are not matched by similar gains in subsequent steps generate a bottleneck. This can be avoided by generating intermediate hold points, which allow for the decoupling of operations and adjusting operation loads to their respective capacity. Storing intermediates at low temperatures (e.g. −20 to −80° C.) is a typical procedure at the laboratory scale and can only be transferred to large scale manufacturing provided the involved product amounts and volumes are sufficiently small. However, for processes of high productivity, storing the required large volumes of frozen materials can be expensive and inconvenient. Furthermore, shipping large volumes of frozen intermediates to other locations for drug processing also presents a challenge.

In addition to the problems associated with storing and shipping intermediates in the biologic manufacturing process, the variety of purification techniques that are used to purify biologics with different characteristics can also decrease the efficiency of the overall manufacturing process. For example, numerous purification techniques including size exclusion chromatography, ion exchange chromatography, affinity chromatography and high performance liquid chromatography (HPLC) can be used to produce a particular protein, and therefore, the BDS for any one protein therapeutic may be substantially different from the BDS for another protein therapeutic. For example, the BDS products may differ in physical state, purity, solubility, type of contaminants, buffers present etc., and each of these characteristics can alter the steps that are necessary for drug product manufacturing (i.e. the conversion of the bulk drug product to a pharmaceutically acceptable product). As a result, the steps involved in the drug product manufacturing are frequently specifically designed for each individual protein purification process depending upon the particular characteristics of the final bulk protein product. Thus, converting the BDS into a form that can be used for drug product manufacturing can also be a bottleneck in the overall protein pharmaceutical formulation scheme.

The present invention is directed towards overcoming these potential bottlenecks. In particular, this can be accomplished through the use of a stable storage intermediate that allows for storage in decreased volumes, at practical temperatures and in a uniform storage form. The present invention provides flexibility to biologics manufacturing by providing a stable and easy to handle storage intermediate. The formation of the stable storage intermediate also allows the decoupling of upstream and downstream process during biologic manufacturing can also be accomplished.

### BRIEF SUMMARY OF THE INVENTION

The present invention is directed to a process of manufacturing a biologic. The process can comprise culturing cells that produce a biologic; harvesting the biologic from the cell culture; forming a stable storage intermediate and storing the intermediate; and further purifying or processing the intermediate. In some embodiments, the further processing or purifying comprises chromatography, filtration, viral inactivation or lyophilization. The process can further comprise forming a bulk drug substance. In addition, the process can still further comprise forming a drug product from the bulk drug substance. The process can also comprise administering the drug product to a patient in need thereof.

In some embodiments of the invention, the storage is for at least 10 days and at a temperature above about −50° C. In other embodiments, the storage is for at least three weeks, and in yet other embodiments, the storage is for 75 days.

In some embodiments of the invention, the formation of the stable storage intermediate provides at least a ten fold reduction in volume. In other embodiments, the formation of the stable storage intermediate provides at least a twenty fold reduction in volume. In some embodiments of the invention, the stable storage intermediate is formed from at least about 500 liters. In another embodiment, the stable storage intermediate is formed from at least about 1,000 liters. In some embodiments, of the invention the cells are cultured in a particular volume, for example, in a volume of of at least about 1,000 liters or at least about 10,000 liters.

In some embodiments, the cells are cultured in a bioreactor. In some embodiments, the harvesting is achieved by centrifugation.

In some embodiments of the present invention, the stable storage intermediate is a solid, semi-solid or suspension. In some embodiments, the solid, semi-solid or suspension is a liquid, frozen liquid, crystal, precipitate, freeze-dried formulation, lyophilized formulation, powder, vesicle or microsphere.

In one embodiment of the invention, the stable storage intermediate is formed by phase separation. In one particular embodiment of the invention, the stable storage intermediate is formed by precipitation. For example, the stable storage intermediate is formed by precipitation with PEG or by precipitation with PEG and zinc.

In some embodiments of the invention, less than about 95% of the content of the stable storage intermediate is the biologic. In another embodiment, about 50-95% of the content of the stable storage intermediate is the biologic.

In some embodiments of the invention, the stable storage intermediate has a low salt concentration. In another embodiment of the invention the stable storage intermediate comprises a protein of high concentration. In yet another embodiment, the stable storage intermediate prevents aggregation of the biologic.

In some embodiments of the invention, the stable storage intermediate increases the stability and/or shelf-life of the biologic. In one embodiment of the invention, the stable storage intermediate is stable for at least 30 days. In another embodiment of the invention, the stable storage intermediate is stable for at least 75 days. In yet another embodiment, the stable storage intermediate is stable for at least three months. In still another embodiment, the stable storage intermediate is stable for at least four months.

In some embodiments of the invention, the stable storage intermediate is stable at 2-8° C. In other embodiments of the invention, the stable storage intermediate is stable at −20° C.

According to the present invention, the biologic can be a protein, metabolite, polypeptide or polynucleotide. In some particular embodiments, the biologic is an antibody.

In some embodiments of the invention, the formation of a drug product comprises using sterile filtration, chromatography and/or ultrafiltration/diafiltration to form a product; filling the product into a container; and optionally, lyophilizing the product. In one particular embodiment, the container is a vial, a syringe or an autoinjector.

In addition, the present invention is also directed to a biologic manufacturing process comprising means for harvesting a product from a bioreactor process; and means for forming a stable storage intermediate from the harvested product; wherein formation of the stable storage intermediate decouples the upstream bioreactor process from a downstream purification and/or drug manufacturing process.

The present invention relates to decoupling upstream bioreactor processes from downstream purification steps by producing a stable storage intermediate directly from the upstream bioreactor processes (e.g., post harvest or post Protein-A). This intermediate can be stored when it is convenient, or when throughput and capacity can be more equally matched, fed into a downstream process of fixed throughput and capacity in variable amounts. By the invention, downstream purification processes do not need to be redesigned every time bioreactor productivity is altered. In addition, the present invention also provides for producing a stable storage intermediate directly from the downstream purification steps, prior to the final drug product formulation.

Decoupling upstream and downstream processes, according to the present invention, allows for manufacturing flexibility. The decoupling can be achieved by providing a means for storing process intermediates that contain a product of interest. Such intermediates can then be subject to purification steps, such as ultrafiltration or diafiltration, to purify the product. The purified product, according to the present invention, can also be formulated by providing a means for storing the purified product of interest in a stable storage intermediate.

Suitable means for generating stable storage intermediates include, e.g., crystals, precipitates, freeze-drying, and microspheres. According to the invention, such means result in a phase separation of the protein or purified protein product away from other components generated by the upstream bioreactor process or by the downstream purification steps. Stable storage intermediates, according to the present invention, allow for a reduced product volume for storing the protein or purified protein product, as compared to the volume of product obtained by traditional purification methods, e.g., centrifugation or chromatography. Stable storage intermediates, according to the present invention, also enhance the stability or shelf life of the protein or purified protein product.

In certain embodiments of the invention, microparticle formation is used as a means for generating a storage intermediate containing protein. In some particular embodiments, a narrow distribution (1-4 um) of protein microspheres with greater than 90% of the microsphere content being protein are generated.

In another embodiment, crystals are used as a means for generating a storage intermediate. In still another embodiment, precipitation is used as a means for generating a stable storage intermediate. In a further embodiment, freeze-drying is used as a means for generating a stable storage intermediate. In certain other embodiments, lyophilization is used as a means for generating a stable storage intermediate.

In additional embodiments of the invention, the stable storage intermediate is formed by phase separation.

In a further aspect, the method of the invention further comprises manufacturing a drug product from the stable storage intermediate. In certain embodiments, this manufacturing step comprises (1) further purifying the product using sterile filtration, chromatography and/or ultrafiltration/diafiltration; (2) filling the product into a container; and (3) optionally, lyophilizing the product.

In a further aspect of the invention, the method comprises forming a second stable storage intermediate after the step of further purifying the product using sterile filtration, chromatography and/or ultrafiltration/diafiltration.

In further embodiments, the microsphere is microparticle. In certain other embodiments, the microparticle is made using water-soluble polymers to co-precipitate the product into the stable storage intermediate.

In further embodiments of the invention, at least 90% of the content of the stable storage intermediate is the product.

According to the present invention, a stable storage intermediate prevents aggregation of the product, prevents an increase in solution viscosity of the product, reduces the volume of the isolated product and/or increases the stability and/or shelf-life of the product.

In a further embodiment, the stable storage intermediate is stable for at least three months at 2°-8° C.

The present invention also provides for a product formulation development process comprising: means for harvesting cells from a bioreactor process; and means for forming a stable storage intermediate from the harvested cells; wherein formation of the stable storage intermediate decouples the upstream bioreactor process from a downstream purification and/or drug manufacturing process. In additional embodiments, the product formulation development process comprises means for further purifying the product using sterile filtration, chromatography and/or ultrafiltration/diafiltration; and means for forming a second stable storage intermediate after further purifying the product using sterile filtration, chromatography and/or ultrafiltration/diafiltration.

### BRIEF DESCRIPTION OF THE DRAWINGS/FIGURES

FIG. 1. shows an example of a process for manufacturing a biologic and displays the steps undertaken in (1) bulk drug substance (BDS) manufacturing and (2) drug product (DP) manufacturing.

FIG. 2. shows an example of a process for manufacturing a biologic and highlights steps at which decoupling may be useful. Dotted circles indicate steps after which decoupling by forming a stable storage intermediate may be useful.

FIG. 3. shows the results of size exclusion chromatography analysis on stable storage intermediates of monoclonal antibodies produced by precipitation and stored at −20° C. (a) and 2-8° C. (b). Precipitated storage intermediates were resuspended and analyzed at time 0, 1 day, 30 days, 75 days, and 135 days for percent monomer, percent high molecular weight 1, percent high molecular weight 2, and percent low molecular weight.

### DETAILED DESCRIPTION OF THE INVENTION

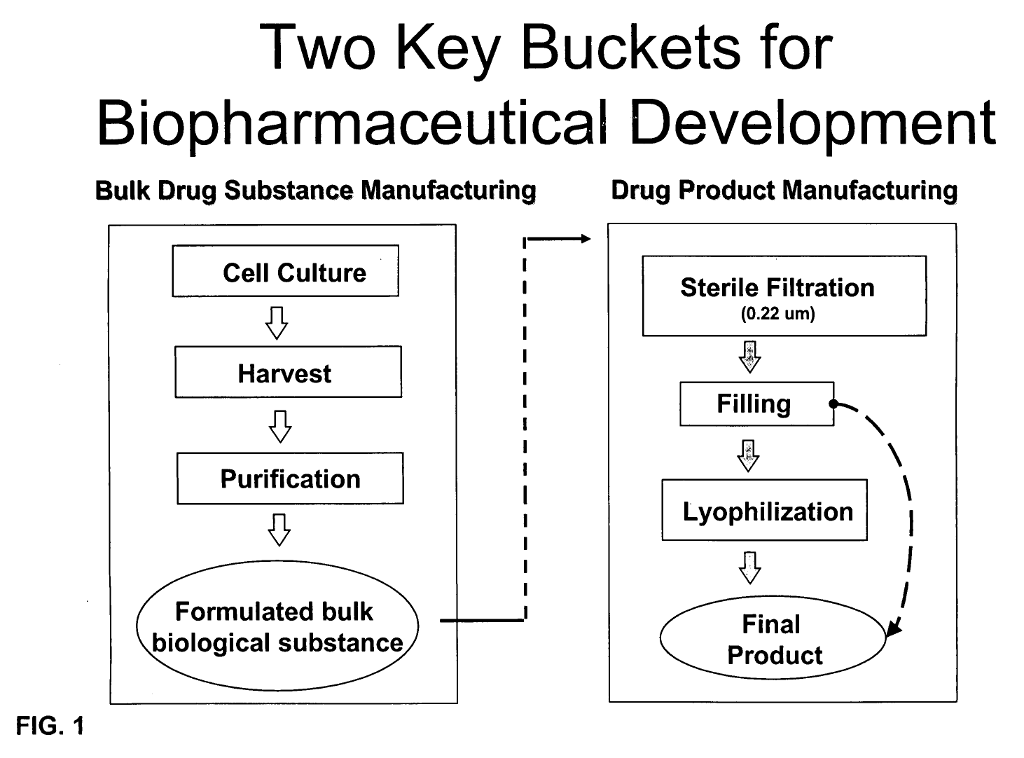

The present invention is directed to increasing the flexibility of industrial-scale manufacturing of biologics by providing a stable and easy to handle storage intermediate. Typical industrial-scale processes for producing biologics can be divided into two main steps: (1) purifying a biologic from a cell culture to produce a bulk drug substance (BDS); and (2) manufacturing a BDS into a drug product (DP). (FIG. 1 provides an example of the steps that may be involved in a typical industrial-scale manufacturing process.) Purification of the BDS from the cell culture typically requires a series of purification steps that occur over a period of several days. Frequently, the BDS represents the first long term hold point in this series of uninterrupted operations. BDS is produced in various physical states, e.g. as a frozen liquid (−20° C. to −70° C.), a liquid stored at a refrigerated temperature (e.g. 2-8° C.), or in a lyophilized form. In this state, it can be stored for varying time periods, and is sometimes shipped to another location, before it is converted to the final DP. However, according to the present invention, the formation of a stable storage intermediate during the industrial-scale process prior to the BDS stage allows for several advantages including, a decreased storage volume, a decreased need for freezing in storage, an increased ability to provide a uniform storage intermediate and an increased ability to pause between manufacturing steps as necessary.

### Stable Storage Intermediates

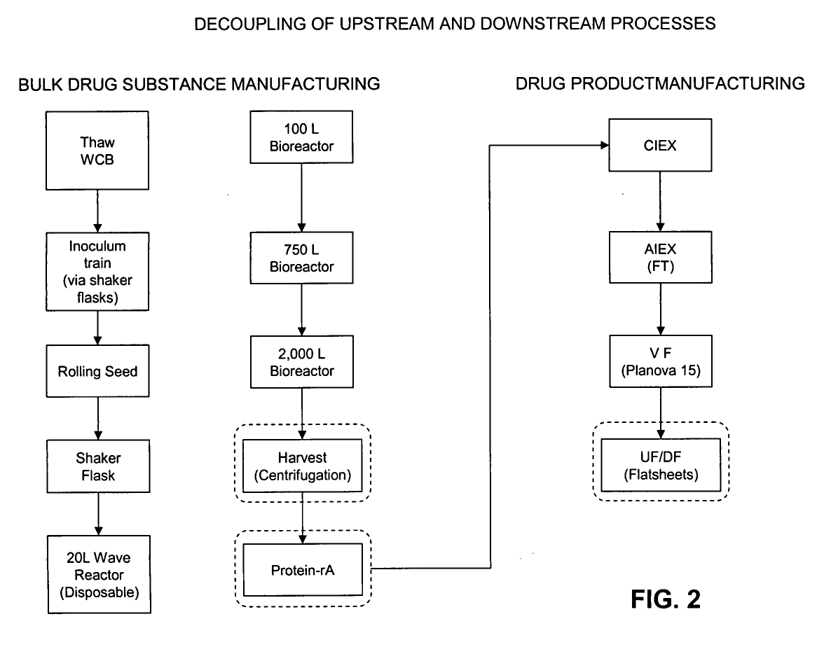

The stable storage intermediate of the present invention can be formed at any step of the large-scale manufacturing process. In some embodiments of the invention, the stable storage intermediate is formed before the formation of the bulk drug substance. FIG. 2 highlights several steps of a manufacturing process at which stable storage intermediates may be useful. According to the present invention, use of a stable storage intermediate before the formation of the BDS can improve the efficiency and flexibility of the overall biologic manufacturing process. Stable storage intermediates can be used between any of the steps within BDS manufacturing and/or between any of the steps within DP manufacturing. For example, the storage stable intermediate can be formed immediately after harvesting a biologic from a cell culture. The storage stable intermediate can be formed after a first purification step, or a subsequent purification step. The storage stable intermediate can be formed after a protein A step, a chromatography step and/or a filtration step. The storage stable intermediate can be formed after an ultrafiltration and/or a diafiltration step. Thus, according to the present invention, a stable storage intermediate can be used between any steps of BDS manufacturing, between BDS manufacturing and DP manufacturing steps, between any steps of DP manufacturing or in any combination of such steps. In one particular embodiment of the invention, the stable storage intermediate can be formed during BDS manufacturing, i.e. before the formation of the BDS.

The storage intermediates of the present invention are particularly useful because they are stable and thereby allow increased flexibility in the manufacturing processes by allowing for extended pauses at desired times within the process. In particular embodiments of the invention, the intermediate is stable for at least two weeks, for at least three weeks, for at least thirty days, for at least a month, for at least 75 days or for at least 135 days.

The stable storage intermediates of the present invention are also particularly useful because they do not need to be maintained at −80° C. or −50° C. For example, the storage stable intermediates of the invention can be stable at temperatures of about −50° C., −40° C., −30° C., −20° C., −10° C., −5° C., 0° C. In some embodiments, the stable storage intermediates are stable at temperatures from about −50° C. to −40° C., −40° C. to −30° C., −30° C. to −20° C., −20° C. to −10° C., −10° to −5° C., −5° C. to 0° C. or 0° C. to 25° C. In some embodiments, the storage stable intermediates of the invention can be stable at temperatures of about −50° C. to 25° C., −40° C. to 25° C., −30° C. to 25° C., −20° C., to 25° C., −10° to 25° C., 5° C. to 25° C. or 0° C. to 25° C. In some embodiments, the stable storage intermediates are stable at room temperature (25° C.). In some embodiments, the stable storage intermediate is stable at room temperature for at least two weeks, for at least three weeks, for at least thirty days, for at least a month, for at least 75 days or for at least 135 days. In some embodiments, the stable storage intermediate is stable at a refrigerated temperature (2-8° C.). In some embodiments, the stable storage intermediate is stable at 2-8° C. for at least two weeks, for at least three weeks, for at least thirty days, for at least a month, for at least 75 days or for at least 135 days. In some embodiments, the stable storage intermediate is stable at −20° C. In some embodiments, the stable storage intermediate is stable at −20° C. for at least two weeks, for at least three weeks, for at least thirty days, for at least a month, for at least 75 days or for at least 135 days.

In some embodiments of the present invention, the stable storage intermediate is stable in air and does not need to be stored under vacuum. However, the stable storage intermediate can optionally be stored under vacuum. In some embodiments of the present invention the stability of the stable storage intermediate is increased upon storage under vacuum. In some embodiments, the stable storage intermediate is stable from at least 50%, 40%, 30%, 20%, 10%, or 5% relative humidity for at least two weeks, for at least three weeks, for at least thirty days, for at least a month, for at least 75 days or for at least 135 days.

The stable storage intermediate of the invention can be a solid, a semi-solid or a suspension. The solid, semi-solid or suspension can be a liquid, frozen liquid, crystal, precipitate, freeze dried particles or microspheres, lyophilized formulation (lyophile), powder, vesicle or microparticle (e.g., a microsphere). In one particular embodiment, the stable storage intermediate is in the form of a powder. In another particular embodiment, the stable storage intermediate is in the form of a precipitate.

In further embodiments of the invention, the stable storage intermediates result in a reduced volume of storage. For example, the volume can be reduced at least about 2 fold, at least about 5 fold, at least about 10 fold, at least about 20 fold, at least about 30 fold, at least about 40 fold, at least about 50 fold, at least about 100 fold, at least about 500 fold, at least about 1,000 fold, or at least about 10,000 fold from the volume prior to the formation of the stable intermediate.

In some embodiments of the present invention, the stable storage intermediate has a low salt concentration. Stable storage intermediates with low salt concentrations are particularly useful in manufacturing processes where downstream processes are salt-sensitive. In the specific case of a biologics manufacturing process, intermediates with high salt concentrations can decrease the efficiency of downstream processing steps. For example, ion exchange chromatography is known to require a low salt concentration during product loading since the capacity of ion exchangers is negatively correlated to the ionic strength of the load solution. In some embodiments of the invention, the low salt concentration of the stable storage intermediate allows a flexible choice in the purification operation carried out subsequent to storage. In some particular embodiments, the salt concentration of the stable storage intermediate is less than about 5 M, 2.5 M, 1 M, 500 mM, 400 mM, 300 mM, 200 mM, 100 mM, 50 mM, 25 mM, 10 mM or 5 mM.

In some embodiments of the invention, the stable storage intermediate has a particular purity. For example, stable storage intermediates formed at different steps in the manufacturing process are expected to have different levels of product purity and different characteristics. For example, stable storage intermediates generated post-harvest can have a 30-50% product purity, while post purification (i.e. protein A column purification) stable storage intermediates can have 90% product purity and post-UF/DF stable storage intermediates can have about or at least 99% product purity. Therefore, according to one embodiment of the invention, the stable storage intermediate contains a biologic that is about 20-90%, 20-80%, 20-70%, 20-60% or 20-50% pure. In another embodiment, the stable storage intermediate contains a biologic that is 30-90%, 30-80%, 30-70%, 30-60% or 30-50% pure. In yet another embodiment, the stable storage intermediate contains a biologic that is 40-90%, 40-80%, 40-70%, 40-60% or 40-50% pure. In some embodiments, the stable storage intermediate is less than about 99%, 98%, 97%, 96%, 95%, 90%, 85%, 80%, 75%, 70%, 65%, 60%, 55% or 50% pure. In yet another embodiment, the biologic is about 70-99%, 75-99%, 80-99%, 85-99%, 90-99%, 70-95%, 75-95%, 80-95%, 85-95%, 90-95%, 70-90%, 75-90%, 80-90%, 85-90%, 70-85%, 75-85% or 80-85% pure. In one embodiment, the stable storage intermediate contains a biologic that is about 90% pure.

In some embodiments, the stable storage intermediate contains a particular concentration of a contaminant, such as a salt, a polymer, other host cell proteins etc. For example, the stable storage intermediate may contain no more than about 25%, 20%, 15%, 10%, 5%, 4%, 3%, 2% 1% or 0.05% of a particular contaminant.

In some embodiments, the choice of stable storage intermediate can be determined by the type of final drug product desired. For example, liquid or frozen liquid stable storage intermediates may be useful for the production of final drug products that are liquids. Frozen liquid stable storage intermediates may also be useful for the formation of final drug products that are lyophilized for frozen liquid final drug products. In particular, the effect of the type of stable storage intermediate and the type of final drug product on storage, stability, shelf-life and presentation of the stable storage intermediate and the final drug product should be considered.

Certain stable storage intermediates of the invention can be compatible with various types of products, including but not limited to, peptides up to 5 kD, low molecular weight proteins (for example, proteins, 10-30 kDs), high molecular weight proteins (for example 50-150 kDs) such as monoclonal antibodies and binding proteins). The proteins or polypeptides of the invention can be cytokines, hormones, clotting factors, growth factors, antibodies, antigenic peptides for vaccines, fragments or derivates of such polypeptides etc. In other embodiments of the invention, the stable storage intermediates of the invention can be compatible with nucleic acids including antisense, oligonucleotides, siRNAs, DNA, or small molecules.

Certain stable storage intermediates of the invention provide various advantages, in addition, to decoupling upstream and downstream processes in manufacturing. For example, some storage stable intermediates allow for prolonged storage of intermediates that can, for example, improve shipment capabilities or the shelf-life of, for example, the bulk drug substance. Some particular stable storage intermediates of the invention allow for high concentration protein storage. Certain stable storage intermediates of the invention increase manufacturing flexibility and/or efficiency. Furthermore, stable storage intermediates of the invention may increase the effectiveness of protein purification steps. In addition, stable storage intermediates of some embodiments of the invention prevent aggregation of a product or protein, and in particular a protein of high concentration. Certain stable storage intermediate also prevent an increase in solution viscosity of the isolated BDS and/or DP.

In some embodiments of the invention, the form of the stable storage intermediate can be the same, or a similar form that is used in the final drug formulation product. For example, post-harvest protein may be stored as an intermediate in a storage type that is also used in a final formulation, for example, as a high-concentration protein microsphere or protein crystal suspension.

### Formation of a Stable Storage Intermediate

Suitable means for generating a stable storage intermediate include, for example, phase separation techniques, e.g. precipitation, crystallization, lyophilization, freeze-drying and microparticle formation.

In one embodiment, precipitation is used as a means for generating a storage intermediate. Accordingly, precipitation can be used to remove a soluble biologic of interest from a solution into a solid phase (a precipitate) to form a stable storage intermediate. Precipitation techniques are well known in the art. Precipitation can be achieved, for example, by altering salt concentrations, adding or removing organic solvents, changing the pH, adding multivalent metal ions, adding nonionic polymers or shifting the temperature. Any protein precipitation means that results in a stable storage intermediate with increased stability, shelf-life and/or reduced storage volume can be used according to the present invention.

In some particular embodiments of the present invention, precipitation using a salt is used to form the stable storage intermediate. The salt can contain, for example, citrate, phosphate, sulfate, acetate, chloride, nitrate or thiocyanate. In some embodiments, ammonium sulfate is used to form a precipitate.

In some particular embodiments, precipitation by adjusting the pH is used to form the stable storage intermediate. For example, the pH can be adjusted to about 7, or adjusted to the isoelectric point of the biologic of interest in order to form the stable storage intermediate. In some embodiments, the pH is adjusted to about 5.5-8.5, 6-8, 6.5-7.5, 6.5-7.0 or 7.0 to 7.5. In some embodiments, the pH is adjusted to about 7.2.

The pH can also be adjusted by various means. In some embodiments, the pH is lowered by addition of an acid. Suitable acids include, but are not limited to, strong acids such as perchloric acid (HClO₄), hydroiodic acid (HI), hydrobromic acid (HBO, hydrochloric acid (HCl), nitric acid (HNO₃), sulfuric acid (diprotic) (H₂SO₄), or weak acids such as acetic acid (CH₃COOH) (e.g., glacial acetic acid), citric acid (C₆H₈O₇), formic acid (HCOOH), hydrocyanic acid (HCN), hydrogen sulfate ion (HSO₄⁻) or combinations of any of the acids listed above. In some embodiments, the pH can be adjusted by use of buffers, such as phosphate buffers (e.g., sodium and potassium phosphates), bicarbonate buffers, citrate buffers, borate buffers, acetate buffers, tromethamine buffers, HEPES buffers and combinations thereof. In one embodiment, the pH is adjusted using Tris base.

In some particular embodiments, precipitation by the addition of metal ions is used to form the storage stable intermediate. For example, Mn²⁺, Fe²⁺, Ca²⁺, Mg²⁺ or Ag⁺ can be used to precipitate the biologic. In some particular embodiments, precipitation by the addition of organic solvents is used to form the stable storage intermediate. For example, ethanol can be used to precipitate the biologic.

In some particular embodiments, precipitation by the use of polymers and/or polyelectrolytes can be used to form the storage stable intermediate. For example, PEG (polyethyleneglycol), dextran, other water-soluble polymers, polyacrylic acid, carboxymethlcellulose or polythyleneimines can be used to precipitate the biologic.

In some embodiments, the concentration of PEG used for precipitation can be up to about 20%, 15%, 10%, 5%, 4%, 3% or 2% PEG (w/w). The PEG solution used for precipitation can also be about 0.05-20%, 0.05-15%, 0.05-10%, 0.05-5%, 0.05-4%, 0.05-3% or 0.05-2% PEG (w/w). The PEG solution used for precipitation can also be about 0.10-20%, 0.10-15%, 0.10-10%, 0.10-5%, 0.10-4%, 0.10-3% or 0.10-2% PEG (w/w). The PEG solution used for precipitation can also be about 0.50-20%, 0.50-15%, 0.50-10%, 0.50-5%, 0.50-4%, 0.50-3% or 0.50-2% PEG (w/w). The PEG solution used for precipitation can also be about 1-20%, 1-15%, 1-10%, 1-5%, 1-4%, 1-3% or 1-2% PEG (w/w). In one embodiment, the PEG solution used for precipitation can be about 5-10% PEG (w/w). In one embodiment, the PEG solution used for precipitation can be about 1.5% PEG (w/w).

The PEG can have a molecular weight of about 400-35,000. For example, in one embodiment, the PEG has a molecular weight of at least about 500. In another embodiment, the PEG has a molecular weight of less than about 20,000 or 10,000. In another embodiment, the PEG has a molecular weight of about 400 to 2,000; 2,000 to 5,000; 5,000 to 10,000; or 10,000 to 35,000. In another embodiment, the PEG has a molecular weight of about 1000-10,000; 2,000 to 8,000; 3,000 to 6,000; or 3,000 to 5,000. In yet another embodiment, the PEG has a molecular weight of 3,000 to 4,000; 2,000 to 6,000; or 1,000 to 7,000. In one particular embodiment, the PEG has a molecular weight of about 3350.

In another specific embodiment of the invention, the addition of zinc is used to precipitate the biologic and form the stable storage intermediate. The concentration of zinc can be up to about 100 mM, 75 mM, 50 mM, 25 mM, 20 mM, 15 mM, 10 mM, 5 mM, 4 mM or 3 mM. The concentration of zinc can also be about 0.5-100 mM, 0.5-75 mM, 0.5-50 mM, 0.5-25 mM, 0.5-20 mM, 0.5-15 mM, 0.5-10 mM, 0.5-5 mM, 0.5-4 mM or 0.5-3 mM. The concentration of zinc can also be about 1-10 mM, 1-7.5 mM, 1-5.0 mM, 1-3 mM or 1-2.5 mM. In one embodiment, the zinc is about 2.5 mM.

In some specific embodiments a combination of precipitation techniques is used.

For example, precipitation can occur by the addition of salt and the decrease in temperature, or by the change of pH and the addition of an organic solvent. In one particular embodiment, the precipitation occurs by the addition of a polymer and an ion. In yet another embodiment, precipitation from a PEG and zinc solution is used as means for generating a stable storage intermediate. In some embodiments, a PEG and zinc solution contains about 1.5% PEG and about 2.5 mM zinc.

In one embodiment production of microspheres is used as a means for generating a stable storage intermediated. As used herein, the term “microspheres” refers generally to microparticles, microspheres and beads. Microspheres are known in the art and can be made of synthetic polymers, natural polymers, copolymers, proteins and/or polysaccharides. They have been used in a variety of applications including protein purification techniques, drug delivery technology and diagnostic applications. According to the present invention they can also be used to generate a stable storage intermediate. The microspheres of the present invention can be in the form of a powder.

In some embodiment, the microspheres are used to generate a means for a storage intermediate containing a high protein concentration. The protein content of the microspheres can be greater than 70%, 80%, 90% or 95% of the microsphere by weight.

In some embodiments, the microspheres have a particular size distribution. For example, the microspheres can be formulated to have a diameter of 1-5 microns, 1-50 microns, or 1-100 microns. In some embodiments the microspheres are greater than 90% protein drug and have a narrow size distribution, for example 1-50 microns in diameter.

In one particular embodiment, the microspheres of the invention can be produced by combining a macromolecule and a polymer in an aqueous solution at a pH near the isoelectric point of the macromolecule and exposing the solution to an energy source for a sufficient amount of time to form microparticles. For example, in one embodiment, the microparticles of the invention are the produced using PROMAXX™ microsphere technology. The PROMAXX™ technology is designed to provide sustained release pharmaceutical formulations and is described in more detail in U.S. Pat. Nos. 5,554,730; 5,578,709; 5,981,719; 6,090,925; and 6,268,053 which are herein incorporated by reference in their entirety.

In another embodiment, crystals are used as a means for generating a storage intermediate that can be used to decouple upstream and downstream process in manufacturing. Techniques for crystal formation are known in the art. For example, methods of preparing protein crystals are described in Protein Crystallization: Techniques, Strategies, and Tips: A Laboratory Manual (Bergfors, Internat'l University Line (1999)). Protein crystallization technologies are also described in more detail in U.S. Pat. Nos. 6,359,118 and 6,500,933, which are herein incorporated by reference in their entirety. Any crystallization means that results in a stable storage intermediate with increased stability, shelf-life and/or reduced storage volume can be used according to the present invention.

In an additional embodiment, freeze-drying is used as a means for generating a stable storage intermediate. In another embodiment, lyophilization is used as a means for generating a stable storage intermediate. Techniques of freeze-drying and lyophilizing proteins are well know in the art and are described, for example, in Freeze-Drying/Lyophilization of Pharmaceutical and Biological Products (Rey and May, 2^{nd} edition, Informa Health Care (2004)). Any freeze-drying or lyophilization means that result in a stable storage intermediate with increased stability, shelf-life and/or reduced storage volume can be used according to the present invention.

### Manufacturing Processes for Biologics

Referring to FIGS. 1 and 2, a process for manufacturing biologics involves a bulk drug substance (BDS) manufacturing step whereby living cells are cultured using an industrial scale culturing system, such as a bioreactor system. Products of the cultured cells are harvested and then can be purified to form a BDS. A “bulk drug substance” or “BDS” refers to a composition, formulation, suspension or solution containing a product, where the product is produced by cells cultured in an industrial-scale process, harvested from the cells and then at least partially purified. Generally, the BDS must been particular release specifications that include quality attributes, strength, purity, identity and safety assessments. In some embodiments of the invention, the stable storage intermediate is formed after harvesting the biologic, but before formation of the BDS.

After formation of the BDS, the next activity is DP manufacturing. In DP manufacturing, the BDS can be for example sterile filtered. After sterile filtration, the product can be filled in a container or package and presented as a final product. Optionally, the product can also be lyophilized, for example, after the filling step, and this lyophilized product can then be presented as a final product.

FIGS. 1 and 2 are intended to provide an exemplary outline of a process for manufacturing biologics. However, manufacturing processes of the invention are not limited to those that follow the particular steps as described in FIGS. 1 and 2. For example, the manufacturing processes of the present invention do not necessarily have to include most or all of the steps as described in the figures. Additionally the steps in the manufacturing processes of the invention may occur in a sequence that is different than that shown in the figures and may include other steps not shown in the figures or explicitly described in the specification.

### Use of Stable Storage Intermediate in a Manufacturing Process for Biologics

As described above, the present invention relates to a stable storage intermediate to be used in industrial-scale biologic manufacturing, and the stable storage intermediate can be formed at any stage of the manufacturing process. Use of the stable storage intermediate provides flexibility to the manufacturing process by decoupling upstream and downstream manufacturing activities, for example, by decoupling the upstream bulk drug substance (BDS) manufacturing activity from the downstream BDS manufacturing activities. Decoupling can occur, for example, after harvesting the product from the bioreactor process, after purification of the harvested product, and/or after filtration steps. Decoupling can also occur during drug product formation.

“Decoupling” refers to providing for a stable storage intermediate at any stage within the process for manufacturing the biologic. For example, decoupling can occur during the bulk drug substance manufacturing, such as post-harvesting or post-purification. Decoupling can also occur during the drug product (DP) manufacturing activity, e.g. after further purification of the BDS (e.g., post-ultrafiltering (UF)/diafiltering (DF)). Decoupling can also occur between the bulk drug substance manufacturing and the drug product manufacturing. “Decoupling” also refers to providing for a stable storage intermediate in which the harvested, purified, or prepared bulk product are prepared as a stable storage intermediate. Decoupling is accomplished by the creation of a stable storage intermediate that adds flexibility to the process of manufacturing a biologic product. Thus, “decoupling means” refers to means for reducing or eliminating the coupling of one activity to another, means for separating, detaching, disconnecting, or equivalents thereof. Decoupling means include any means of forming a stable storage intermediate as described above, including for example, precipitation, crystallization and freeze-drying.

Decoupling can allow for an extended pause or storage stage in the biologic manufacturing process. For example, once a stable storage intermediate is formed, it can be stored for at least about 10 days, at least about two weeks, at least about three weeks, at least about a month, at least about two months, at least about six months or at least about a year. The storage does not need to be at −80° C. For example, the stable storage intermediate of the present invention can be stored at 2-8° C. or at −20° C. The stable storage intermediate can be stored at above about −40° C., −30° C., −20° C., -10° C., −5° C., 0° C. The stable storage intermediate can be stored at about 4° C. or at room temperature. The term “storage” refers to keeping, maintaining or holding the intermediate for a period of time in a relatively constant state without manipulation. The stored form can be moved between locations and is still considered stored as long as the intermediate has not been materially altered or processed.

According to the present invention, the stable storage intermediate is then used in downstream processing steps to form the bulk drug substance. Depending on the form of the stable storage intermediate and the storage conditions, the intermediate may need to be thawed, reconstituted, resuspended and/or mixed before downstream processing steps can begin. The stable storage intermediate can be reconstituted or resuspended in any media or buffer appropriate for downstream processing steps and can be reconstituted or resuspended in a volume that is smaller than, equivalent to or larger than the volume from which the stable storage intermediate was formed. In one embodiment the stable storage intermediate is reconstituted in a volume that is at about one half, one quarter, or one tenth of the volume from which the stable storage intermediate was formed. In another embodiment, the stable storage intermediate is reconstituted in a volume that is about equivalent to the volume from which the stable storage intermediate was formed. In yet another embodiment, the stable storage intermediate is reconstituted in a volume that is about two, three, four, five, or ten times the volume from which the stable storage intermediate was formed.

In one embodiment, the present invention is directed to a process of manufacturing a biologic comprising culturing cells that produce a biologic, harvesting the biologic from the cell culture and forming a stable storage intermediate. The stable storage intermediate can then be stored for a duration of time and does not need to be frozen at −80° C.

According to one embodiment of the present invention, a biologic manufacturing process of the present invention comprises isolating means, preparing means and decoupling means. The isolating means are for isolating a bulk biological substance from a bioreactor process; the preparing means are for preparing a purified product from the bulk biological substance; and the decoupling means are for decoupling the isolation of the bulk biological substance from the preparation of the purified product, where the decoupling means comprises means for preparing a stable storage intermediate of the bulk biological substance.

The term “isolating means” refers to means for isolating, purifying, separating, extracting, fractionating, precipitating or equivalents thereof. Means for isolating a bulk biological substance are described in more detail below. By an “isolated” protein or metabolite is intended a protein or metabolite that is not in its natural milieu. Various levels of purification can be applied. For example, an isolated polypeptide can be removed from its native or natural environment. Recombinantly produced polypeptides and proteins expressed in host cells can be considered isolated for purposed of the invention, as are native or recombinant polypeptides which have been separated, fractionated, or partially or substantially purified by any suitable technique.

The term “preparing means” refers to means for preparing, producing, isolating purifying or equivalents thereof. Preparing means are described in more detail below.

The term “decoupling means” refers to means for reducing or eliminating the coupling of one activity to another, means for separating, detaching, disconnecting, or equivalents thereof as described above.

In further embodiments of the invention, the process for manufacturing biologics requires: means for isolating a bulk biological substance using a bioreactor process; means for preparing a purified product from the bulk biological substance; and means for decoupling the isolation of the bulk biological substance from the preparation of the purified product based on the preparation of a stable storage intermediate.

### Biologics

A “biologic” as manufactured according to the present invention is produced by cells in an industrial-size manufacturing process. The biologic can be, for example, a biomacromolecule, a protein, a metabolite, a polypeptide or fragment thereof, a polynucleotide or fragment thereof or a small molecule.

The terms “biological biomacromolecule” or “biomacromolecule” as used herein refer to a molecule with a molecular mass exceeding 1 kDa which can be isolated from an organism or from cellular culture, e.g., eukaryotic (e.g., mammalian) cell culture or prokaryotic (e.g., bacterial) cell culture. In some embodiments, the use of the term refers to polymers, e.g., biopolymers such as nucleic acids (such as DNA, RNA), polypeptides (such as proteins), carbohydrates and lipids. In some embodiments, the term “biomacromolecule” refers to a protein. In some embodiments, the term “biomacromolecule” refers to a recombinant protein or a fusion protein. In some embodiments, the protein is soluble. In some embodiments, the biomacromolecule is an antibody, e.g., a monoclonal antibody. Commercially important biomacromolecules include, e.g., proteins and nucleic acids, e.g., DNA and RNA. Two examples of biomacromolecules that are often isolated on an industrial scale are monoclonal antibodies and fusion proteins. These antibodies and fusion proteins are valuable in various diagnostic and therapeutic fields, and have been used to treat various diseases such as inherited and acquired immune-deficiency diseases and infectious diseases.

The biologic product of the present invention can be, for example, a cytokine, hormone, clotting or growth factor, antigenic peptide, antibody or fragment thereof, mRNA molecule, vector or antisense polynucleotide molecule. The protein can be, for example, a therapeutic protein or a protein that recognizes a desired target. The protein can be an antibody. The product or biologic can refer to peptides up to 5 kD; low molecular weight proteins in the range of 10-30 kD; or high molecular weight proteins in the range of 50-150 kD. The product or biologic can be include monoclonal antibodies and polypeptide binding proteins and nucleic acids, including antisense, oligonucleotides, siRNAs and DNA.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

The term “polynucleotide” is intended to encompass a singular nucleic acid as well as plural nucleic acids, and refers to an isolated nucleic acid molecule or construct, e.g., messenger RNA (mRNA) or plasmid DNA (pDNA). A polynucleotide may comprise a conventional phosphodiester bond or a non-conventional bond (e.g., an amide bond, such as found in peptide nucleic acids (PNA)). The term “nucleic acid” refers to any one or more nucleic acid segments, e.g., DNA or RNA fragments, present in a polynucleotide. By “isolated” nucleic acid or polynucleotide is intended a nucleic acid molecule, DNA or RNA, which has been removed from its native environment. For example, a recombinant polynucleotide encoding a TNFα antibody contained in a vector is considered isolated for the purposes of the present invention. Further examples of an isolated polynucleotide include recombinant polynucleotides maintained in heterologous host cells or purified (partially or substantially) polynucleotides in solution. Isolated RNA molecules include in vivo or in vitro RNA transcripts of polynucleotides of the present invention. Isolated polynucleotides or nucleic acids according to the present invention further include such molecules produced synthetically. In addition, polynucleotide or a nucleic acid may be or may include a regulatory element such as a promoter, ribosome binding site or a transcription terminator.

The metabolite can be a substance produced by metabolic action of the cells, for example, a small molecule. A small molecule can have a molecular weight of less than 5,000 Da or less than 1,000 Da. The metabolite can be, for example, a mono- or poly-saccharide, a lipid, a nucleic acid or nucleotide, a peptide (e.g., a small protein), a toxin or an antibiotic.

As used herein, the term “protein” is intended to encompass a singular “protein” as well as plural “proteins.” Thus, as used herein, terms including, but not limited to “peptide,” “polypeptide,” “amino acid chain,” or any other term used to refer to a chain or chains of amino acids, are included in the definition of a “protein,” and the term “protein” may be used instead of, or interchangeably with, any of these terms. The term further includes proteins which have undergone post-translational modifications, for example, glycosylation, acetylation, phosphorylation, amidation, derivatization by known protecting/blocking groups, proteolytic cleavage, or modification by non-naturally occurring amino acids. Proteins also include polypeptides which form multimers, e.g., dimers, trimers, etc. The term protein also includes fusions proteins, e.g., a protein that is produced via a gene fusion process in which a protein (or fragment of a protein) is attached to an antibody (or fragment of antibody). Examples of fusion proteins of the present invention include disulfide-linked bifunctional proteins comprised of linked Fc regions from human IgG1 and human IgE; and lymphotoxin beta receptor immunoglobulin G1.

Antibodies can be biologics according to the present invention. The term “antibody” refers to polyclonal, monoclonal, multispecific, human, humanized or chimeric antibodies, single chain antibodies, Fab fragments, F(ab′)2 fragments, fragments produced by a Fab expression library, anti-idiotypic (anti-Id) antibodies (including, e.g., anti-Id antibodies to antibodies of the invention), and epitope-binding fragments of any of the above. In some embodiments, the term “antibody” refers to a monoclonal antibody. The term “antibody” also refers to immunoglobulin molecules and immunologically active portions of immunoglobulin molecules, i.e., molecules that contain an antigen binding site that immunospecifically binds an antigen. The immunoglobulin molecules that can be purified by the method of the invention can be of any type (e.g., IgG, IgE, IgM, IgD, IgA and IgY), class (e.g., IgG1, IgG2, IgG3, and IgG4) or subclass of immunoglobulin molecule. Antibodies of the present invention also include chimeric, single chain, and humanized antibodies. Examples of antibodies of the present invention include commercialized antibodies, such as natalizmab (humanized anti-a4 integrin monoclonal antibody), humanized Anti-Alpha V Beta 6 monoclonal antibody, humanized anti-VLA1 IgG1 kappa monoclonal antibody; huB3F6 (humanized IgG1/kappa monoclonal antibody).

Antibodies produced and used in accordance with the invention may be from any animal origin including birds and mammals. Preferably, the antibodies purified by the method of the invention are human, murine (e.g., mouse and rat), donkey, ship rabbit, goat, guinea pig, camel, horse, or chicken. As used herein, “human” antibodies include antibodies having the amino acid sequence of a human immunoglobulin and include antibodies isolated from human immunoglobulin libraries or from animals transgenic for one or more human immunoglobulin and that do not express endogenous immunoglobulins. See, e.g., U.S. Pat. No. 5,939,598 by Kucherlapati et al. In some embodiments, the antibody include, but are not limited to, IgG1, IgG2, IgG3, and IgG4 antibodies, including commercialized antobodies, such as natalizmab (TYSBARI®, Elan Pahrmaceuticals, San Diego, Calif.).

Antibodies to be produced and used according to the invention include, e.g., native antibodies, intact monoclonal antibodies, polyclonal antibodies, multispecific antibodies (e.g., bispecific antibodies) formed from at least two intact antibodies, antibody fragments (e.g., antibody fragments that bind to and/or recognize one or more antigens), humanized antibodies, human antibodies (Jakobovits et al., Proc. Natl. Acad. Sci. USA 90:2551 (1993); Jakobovits et al., Nature 362:255-258 (1993); Bruggermann et al., Year in Immunol. 7:33 (1993); U.S. Pat. Nos. 5,591,669 and 5,545,807), antibodies and antibody fragments isolated from antibody phage libraries (McCafferty et al., Nature 348:552-554 (1990); Clackson et al., Nature 352:624-628 (1991); Marks et al., J. Mol. Biol. 222:581-597 (1991); Marks et al., Bio/Technology 10:779-783 (1992); Waterhouse et al., Nucl. Acids Res. 21:2265-2266 (1993)). The antibodies purified by the method of the invention may be recombinantly fused to a heterologous polypeptide at the N- or C-terminus or chemically conjugated (including covalently and non-covalently conjugations) to polypeptides or other compositions. For example, antibodies purified by the method of the present invention may be recombinantly fused or conjugated to molecules useful as labels in detection assays and effector molecules such as heterologous polypeptides, drugs, or toxins. See, e.g., PCT publications WO 92/08495; WO 91/14438; WO 89/12624; U.S. Pat. No. 5,314,995; and EP 396,387.

In some embodiments, the biologic is a soluble protein. The term “soluble” refers to the propensity of a protein to substantially localize to the hydrophilic or aqueous-based environments of a cellular host, e.g., the cytoplasm, periplasm or extracellular medium. Thus, during cellular fractionation, a soluble protein would generally be substantially isolated with the cytoplasmic, periplasmic, or extracellular components of a host cell. In some embodiments, a soluble protein is water soluble in the absence of detergents. One of skill in the art will recognize that neither the cellular localization of a polypeptide, nor the cellular fractionation of a protein, is absolute. Thus, the phrase “substantially localize” refers to a protein in which 50%, 70%, 75%, 80%, 85%, 90%, 95%, or 99% of the protein is in the designated cellular location, e.g., cytoplasm, periplasm, or extracellular medium.

### Bulk Drug Substance Manufacturing

### Cell Culture

According to the present invention, the product or biologic can be produced or expressed by living cells, grown for example in a cell culture. The term “express” or “expression” as used herein refers to a process by which a gene produces a biochemical, for example, a polypeptide or a biomacromolecule. The process includes any manifestation of the functional presence of the gene within the cell including, without limitation, gene knockdown as well as both transient expression and stable expression. It includes without limitation transcription of the gene into messenger RNA (mRNA), and the translation of such mRNA into polypeptide(s). If the final desired product is a biochemical, expression includes the creation of that biochemical and any precursors. Expression of a gene produces a “gene product.” As used herein, a gene product can be either a nucleic acid, e.g., a messenger RNA produced by transcription of a gene, or a polypeptide which is translated from a transcript. Gene products described herein further include nucleic acids with post transcriptional modifications, e.g., polyadenylation, or polypeptides with post translational modifications, e.g., methylation, glycosylation, the addition of lipids, association with other protein subunits, proteolytic cleavage, and the like. The biologic can also be produced by the cells, e.g. a metabolite produced by metabolic action of the cells, for example, a small molecule. The term “produced” includes both “expression” as described above and other methods in which a cell creates the biologic of interest.

The biologic of the present invention can be produced from a cell culture comprising growth media and various eukaryotic cells, e.g., mammalian cells. The mammalian cells of the present invention, including the mammalian cells that are used in the methods of the invention, are any mammalian cells that are capable of growing in culture. Exemplary mammalian cells include, e.g., CHO cells (including CHO-K1, CHO DUKX-B11, CHO DG44), VERO, BHK, HeLa, CV1 (including Cos; Cos-7), MDCK, 293, 3T3, C127, myeloma cell lines (especially murine), PC12, HEK-293 cells (including HEK-293T and HEK-293E), PER C6, Sp2/0, NSO and W138 cells. Mammalian cells derived from any of the foregoing cells may also be used. In one embodiment of the invention, the biologic is produced by CHO cells.

The biologic of the present invention can be produced from a cell culture comprising growth media and various prokaryotic cells, e.g., E. coli, Bacillus subtilis, Salmonella typhimurium and various species within the genera Pseudomonas, e.g., P. aeruginosa, yeast cells, e.g., Saccharomyces, Pichia, Hansenula, Kluyveromyces, Schizosaccharomyces, Schwanniomyces and Yarrowia, insect cells, e.g., Trichoplusia, Lipidotera, Spodoptera, Drosophila and Sf9, or plant cells, e.g., Arabidopsis. One of skill in the art can select an appropriate cell line depending on the particulars of biologic and process of interest. In some embodiments, the cell cultures comprises plant cells.

Cell cultures can be grown and maintained according to any method known in the art, but are generally large-scale cell cultures. For example, in some embodiments the cell culture is at least 500 liters, at least 750 liters, at least 1,000 liters, at least 1,250 liters, at least 1,500 liters, at least 2,000 liters, at least 5,000 liters or at least 10,000 liters.

The term “composition” in the present invention refers to a mixture of one or more molecules of the biologic of the present invention and optionally at least one impurity, wherein the impurity and the biologic are not the same. In some embodiments, the composition comprises a biologic, a cellular host organism (e.g., mammalian cells), and a growth media sufficient for propagating the host organism and allowing expression or production of the biologic of interest.

The selection and use of growth medium are known to those in the art. In some embodiments, the growth media is a cell culture media. Cell culture media vary according to the type of cell culture being propagated. In some embodiments, the cell culture media is a commercially available media. In some embodiments, the composition comprises a growth media which contains e.g., inorganic salts, carbohydrates (e.g., sugars such as glucose, galactose, maltose or fructose) amino acids, vitamins (e.g., B group vitamins (e.g., B12), vitamin A vitamin E, riboflavin, thiamine and biotin), fatty acids and lipids (e.g., cholesterol and steroids), proteins and peptides (e.g., albumin, transferrin, fibronectin and fetuin), serum (e.g., compositions comprising albumins, growth factors and growth inhibitors, such as, fetal bovine serum. newborn calf serum and horse serum), trace elements (e.g., zinc, copper, selenium and tricarboxylic acid intermediates) and combinations thereof. Examples of growth medias include, but are not limited to, basal media (e.g., MEM, DMEM, GMEM), complex media (RPMI 1640, Iscoves DMEM, Leibovitz L-15, Leibovitz L-15, TC 100), serum free media (e.g., CHO, Ham F10 and derivatives, Ham F12, DMEM/F12). Common buffers found in growth media include PBS, Hanks BSS, Earles salts, DPBS, HBSS, EBSS. Media for culturing mammalian cells are well known in the art and are available from, e.g., Sigma-Aldrich Corporation (St. Louis, Mo.), HyClone (Logan, Utah), Invitrogen Corporation (Carlsbad, Calif.), Cambrex Corporation (E. Rutherford, N.J.), JRH Biosciences (Lenexa, Kans.), Irvine Scientific (Santa Ana, Calif.), and others. Other components found in growth media can include ascorbate, citrate, cysteine/cystine, glutamine, folic acid, glutathione, linoleic acid, linolenic acid, lipoic acid, oleic acid, palmitic acid, pyridoxal/pyridoxine, riboflavin, selenium, thiamine, transferrin. One of skill in the art will recognize that there are modifications to growth media which would fall within the scope of this invention.

### Bioreactors

Cell cultures can be grown in a vessel appropriately sized for large-scale manufacture such as a bioreactor. The term “bioreactor” refers to a particular device for culturing living cells (See e.g., WO 2006/071716). The cells can produce or express a desired product, such as a protein or a metabolite.

Generally, a bioreactor cultures cells in a volume of about 1 to 10,000 L or 20,000 L and is equipped with appropriate inlets for introducing the cells and microcarriers, sterile oxygen, various media for cultivation, etc.; outlets for removing cells, microcarriers and media; and means for agitating the culture medium in the bioreactor, such as a spin filter. Exemplary media are disclosed in the art; see, e.g., Freshney, Culture of Animal Cells—A Manual of Basic Techniques, Wiley-Liss, Inc. New York, N.Y., 1994, pp. 82-100. The bioreactor generally also has means for controlling the temperature and preferably means for electronically monitoring and controlling the functions of the bioreactor.

The bioreactor can be, for example, a stirred-tank bioreactor. Such a bioreactor can include a tank holding a liquid medium in which living cells are suspended. The tank can include ports for adding or removing medium, adding gas or liquid to the tank (for example, to supply air to the tank, or adjust the pH of the medium with an acidic or basic solution), and ports that allow sensors to sample the space inside the tank. The sensors can measure conditions inside the bioreactor, such as temperature, pH, or dissolved oxygen concentration. The ports can be configured to maintain sterile conditions within the tank.

The bioreactor can be used for culturing eukaryotic cells, such as a yeast, insect, plant or animal cells; or for culturing prokaryotic cells, such as bacteria. Animal cells can include mammalian cells, an example of which is Chinese hamster ovary (CHO) cells. In some circumstances, the bioreactor can have a support for cell attachment, for example when the cells to be cultured grow best when attached to a support. The tank can have a wide range of volume capacity—from 1 L or less to 10,000 L or more.

Exemplary bioreactor systems, as described in WO 2006/071716, include a vessel that holds a liquid cell culture which can be stirred by an agitator. Conditions inside the vessel are monitored by a plurality of sensors. Sensors can independently provide a measurement as an input to the controller. The controller then compares each input to a setpoint and provides individual outputs. Each output affects the operation of actuators. Operation of each of the actuators, in turn, affects the conditions monitored by sensors. In this way, the control system of sensors, inputs, controller, outputs and actuators serves to maintain the monitored conditions inside the vessel at their setpoints. Sensors can be in contact with the liquid medium or with a headspace gas. The actuators can deliver material to the vessel (for example, an acidic or basic solution, to change the pH of the liquid medium) or can alter other functions of the bioreactor system (such as heating or agitation speed).

Other bioreactor designs are known in the art, and can include, for example, supports for anchorage-dependent culture and/or three dimensional cell culture. The bioreactor can be configured to operate in continuous or a batch mode. In certain examples, a closed fluid circuit, connecting an inlet and an outlet on the bioreactor, is provided for circulation of cells and media and to provide a region for cell separation. Cells and media are circulated from and then to the bioreactor so that the culture process is not disturbed.

A bioreactor can comprises a pump for pumping media to the bioreactor and for pumping cells and media through the fluid circuit; and an inlet port for introducing magnetic particles into the bioreactor for magnetically labeling cells in culture in the bioreactor. The cells are magnetically labeled while in culture, rather than in a buffer or non-native fluid medium. A magnetic separator, which can be located on the fluid circuit, comprises a controllable electromagnet, for separating magnetically labeled cells from circulating media.

The magnetic separator can further comprise a diverter, responsive to the electromagnet, and a collection chamber, attached to the diverter, wherein labeled cells are separated from unlabelled cells on the basis of magnetic labeling, preferably while begin pumped through the fluid circuit, and, again without special separation, re-suspension or rinsing steps.

The separator may be triggered by a separate optical detector, coupled to the magnetic separator, wherein the electromagnet is controlled in response to detection of an optical signal by the optical detector. The optical detector could detect fluorescence from cells that have been dual-labeled with magnets and fluorescent dyes. The optical detector could also be set to be triggered on the basis of size or shape or other properties. A microscope may be used in conjunction with this optical detector, and the cells in the bioreactor may also be examined microscopically.

The bioreactor may further be provided with an electrode that contacts at least a portion of a bioreactor surface adjacent the cultured cells. This electrode may be used to deliver pre-selected pulses of electricity to the cells, so as to cause the cells to adapt into cells having particular electrical activity, e.g., muscle cells. Similarly, the pump used may be a pulsatile pump, which simulates physiological conditions of pumped blood flow, in order to direct cells into certain types of differentiation.

The bioreactor may be adapted for certain specific cell culture and isolation of particularly differentiated cells, and, therefore, may be provided as a kit, which may contain cell culture media, stem cells, and growth and differentiation factors intended to derive cells of specific lineages, such as cells to be used in cardiovascular grafts. The differentiated cells are isolated magnetically, with each pass through the circuit yielding additional cells.

The bioreactor system of the invention is a device in which an inoculum containing cells is filtered so that the cells will pass into and substantially adhere to a three-dimensional porous matrix which serves as the support substrate upon which the cells grow. This support substrate can also function as a filter. The bioreactor also includes channels through which the inoculum may be introduced, and through which fluid media may be introduced for cell nourishment and maintenance.

### Harvesting

Generally, after cell culture, the desired product must be harvested from the cell culture. Harvesting refers to the primary recover of the desired product from the cells producing the product. Harvesting can be accomplished using any method known in the art to separate the desired product from other substances in the cell culture. For example, if the desired product is secreted by the cells in the cell culture, harvesting can be accomplished by centrifugation. After centrifugation the supernatant, which contains the culture media and the secreted product is separated from the pellet which contains the cell bodies and debris. Alternatively, harvesting can be accomplished by microfiltration.

Harvesting generally does not result in a large decrease in volume. In some embodiments the harvest is at least 500 liters, at least 750 liters, at least 1,000 liters, at least 1,250 liters, at least 1,500 liters, at least 2,000 liters, at least 5,000 liters, at least 10,000 liters or at least 18,000 liters. The purity of a product post-harvest varies according to the type of harvesting procedure used. In some embodiments, the purity of the product post-harvest is between about 30-50%.

The term “harvest feed” refers to a media in which cells are present in immediately before harvesting, or a media in which harvested cells are placed immediately after harvesting and into which the cells are resuspended. A harvest feed can include any of materials in growth media, or other media suitable for resuspending the harvested cells or cellular fractions. For example, in some embodiments, the harvest media may contain water, a buffer, osmotic agents, anti-degradation agents, etc.

In some embodiments, it is beneficial or desirable to harvest a biomacromolecule from a high cell density composition (e.g., harvest feed). High cell density compositions present unique problems relative to normal cell density compositions. For example, high cell density compositions can have higher amounts of impurities present in the composition, thereby increasing the amount of impurities that need to be removed during the purification process. Thus, a higher cell density composition can foul a filter more quickly, thereby prohibiting filtration of the composition. In some embodiments, high cell density compositions require the use of more filters, or filters with larger surface areas. Both of these requirements can result in greater costs associated with filtration and/or loss of product. Thus, some embodiments in the present invention are directed to a method of isolating a biomacromolecule present in a high cell density composition. The term “high cell density” generally refers to cell densities in a harvest feed of about 1×10⁵ to 3.5×10⁷, about 1.0×10⁶ to about 1.0×10⁷, or about 5.0×10⁶ to about 9.0×10⁶ cells per ml for mammalian cells. Of course, one of skill in the art will appreciate that various cells traditionally grow at different cell densities. Thus, in some embodiments, “high cell density” cell cultures refers to cell cultures containing cells at a density higher than the density traditionally practiced for that cell line.

Movement of a composition, such as a harvest feed, through a filter during filtration generates a transmembrane pressure resulting from the membrane resistance. As the membrane surface becomes accumulated (or polarized) with cellular material, there is an increased resistance to flow across the membrane at a constant flow rate, thus causing the driving force or transmembrane pressure to increase. If the amount of cellular material near the surface of the membrane is reduced, or if the membrane is less polarized, the transmembrane pressure tends to remain substantially constant. Methods to calculate transmembrane potential are know to those in the art, and include the use of pressure transducers or gauges. In some embodiments of the present invention, the transmembrane pressure can be calculated by taking the difference between the average of the feed and retentate stream outlet pressure and the permeate stream pressure.

According to the present invention, the pH of the composition can be lowered, thereby removing some impurities, and allowing the purification of higher cell density compositions. Generally, during filtration of a composition, e.g., a harvest feed, that has not been pH-adjusted, the transmembrane pressure of a filter increases significantly as more of the composition is loaded onto the filter. For example, in some embodiments the transmembrane pressure increases 5 psi, 7 psi, 10 psi, 15 psi or 20 psi or greater from the start of the filtration process (when the first amount of the composition is placed in the filter) to the end of the filtration process (typically following a 7-10× concentration of cellular material and a 3-5× diafiltration) as the pores of the filter become clogged.

In order to decouple harvesting and downstream purification steps, a stable storage intermediate can be developed post-harvest. Formation of a stable storage intermediate post-harvest can result in successful formation of stable protein precipitates and differential selectivity of the product away from any impurities. Certain storage intermediates post-harvest provide for a reduction in volume of the harvested material, which is beneficial for storage purposes. In addition, the formation of a stable storage intermediate has other benefits: (1) the upstream and downstream processes of the manufacturing process are decoupled; (2) the necessity of a purification steps may be reduced; (3) the downstream purification capability is effectively used; and (4) flexibility and efficiency of the manufacturing process is achieved.

### Purification and Formation of Bulk Biological Substance

Formation of a bulk drug substance also generally also involves a purification process. Purification can be accomplished using any means known in the art, and the purity of the product can vary after initial purification steps. For example, biological macromolecules (i.e., biomacromolecules) such as recombinant biomacromolecules can be purified by many different methods, e.g., filtration, centrifugation, size exclusion chromatography, affinity chromatography, and combinations of the above, just to name a few. The method of purification is generally chosen based on a characteristic of the biomacromolecule that distinguishes it from one or more impurities that coexist with the biomacromolecule in a composition. A vast number of biomacromolecules are commercially important and an ability to purify a large amount of biomacromolecules in a timely and cost effective manner is desired. Extensive research has been performed to increase efficiency of current purification technologies and methods for purifying biomacromolecules. Often, purification techniques that are suitable for small scale preparations are not suitable for industrial-scale purification. In some particular embodiments of the invention, harvesting is followed by filtration, which is followed by formation of a stable storage intermediate. In another particular embodiment, harvesting is followed by protein A purification, which is followed by formation of a stable storage intermediate.

Protein A purification step follows harvesting in many current biological manufacturing processes. The purity of a product after the Protein A purification step (post-Protein A) can be about 90%. Formation of a stable storage intermediate after the Protein A step can result in successful formation of stable protein precipitates and differential selectivity of the product away from any impurities, and provides for stability and reduction in volume, as described above. The formation of a stable storage intermediate after the Protein A purification and/or after other purification steps that occur before drug product manufacturing has benefits as described above including: (1) decoupling of the upstream and downstream manufacturing processes; (2) effective use of the downstream purification capability; and (3) flexibility and efficiency of the manufacturing process.

Purification can also occur by depth filtration. For example, depth filtration is commonly used after centrifugation to remove cellular and other debris. This can aid in the efficiency of downstream purification steps because the debris does not contaminate or clog the later purification steps. In some embodiments, the depth filters are charged depth filters. Charged depth filters are particularly suited to retain large amounts of contaminants using both size exclusion and absorption.

Purification can also involve steps described above that are also used in the formation of a stable storage intermediate. For example, purification steps can involve adjusting the pH, addition of salts, etc. For example, the addition of divalent cations to the composition can also be used in the recovery of the biomacromolecule of interest. Various divalent cations exist and are known to those in the art, and include, e.g., calcium cation (Ca²⁺), magnesium cation (Mg²⁺), copper cation ⁻(Cu²⁺), cobalt cation (Co²⁺), manganese cation (Mn²⁺), nickel cation (Ni²⁺), berylium cation (Be²⁺), strontium cation (Sr²⁺), barium cation (Ba²⁺), radium cation (Ra²⁺), zinc cation (Zn²⁺), cadmium cation (Cd²⁺), silver cation (Ag²⁺), palladium cation (Pd²⁺), rhodium cation (Rh²⁺), and combinations thereof.

One of skill in the art will realize that the cation can exist in salt form, e.g., a calcium salt such as CaCl₂ can produce a calcium cation when placed in an aqueous solution. Thus, as used herein, the phrase “adding a divalent cation” would encompass not only the addition of a cation in its charged stated, but also the addition of a salt or other compound that would produce a divalent cation upon introduction into the composition of the present invention. In some embodiments, the divalent cation is Co²⁺ or Ni²⁺, or their salts (e.g., CoCl₂, NiCl₂, CaCl₂, MnCl₂, MgCl₂, and CuCl₂), or combinations of one or more of these cations or salts. It is to be expected that certain divalent cations may be more suitable for different biomacromolecules. However, one of skill in the art can easily and quickly test many divalent cations to determine which achieves the maximum recovery of the biomacromolecule of interest.

Various concentrations of divalent cations in the composition are suitable for use in the present invention. On of skill in the art will recognize that various amounts of divalent cations are normally present in small amounts in the harvest feed (endogenous divalent cations), and that various amounts of divalent cations can be added to the harvest feed in accordance with the present invention (exogenous divalent cations). In some embodiments, the concentration of the divalent cations comprises both exogenous and endogenous cations. However, for practical purposes, since the amount of endogenous is relatively small compared to the amount of exogenous divalent cations, the concentration of the divalent cations can be calculated by simply considering the exogenous divalent cations. In some embodiments, the divalent cation in the composition is present at a concentration of about 0.01 mM to about 1 M in the composition. In some embodiments, the divalent cation is present at a concentration of about 0.1 mM to about 500 mM, or about 0.5 mM to about 200 mM, about 1.0 mM to about 100 mM, about 2 mM to about 50 mM, about 5 mM to about 15 mM, or about 2 mM to about 20 mM in the composition. In some embodiments, the divalent cation is present at a concentration of about 10 mM in the composition. One of skill in the art will understand that different concentrations of cations may be required for various biomacromolecules.

In addition, traditional approaches to producing purified antibodies include ammonium sulfate precipitation, use of caprylic acid followed by centrifugation, ion exchange chromatography (e.g., DEAE or hydroxyapatite), immunoaffmity purification (e.g., Protein A or Protein G), and dialysis. See e.g., Antibodies: A Laboratory Manual, Harlow and Lane, Cold Spring Harbor Laboratory (1988). The use of a combination of the above methods is common, e.g., antibody purification from plasma using ethanol fractionation followed by ion exchange chromatography and/or caprylic acid (CA) precipitation. See for example McKinney et al., J. Immunol. Methods 96:271-278 (1987); U.S. Pat. Nos. 4,164,495; 4,177,188; RE 31,268; 4,939,176; and 5,164,487. In addition, acidification of fermentation has been used to improve recovery and stability of antibodies and recombinant proteins. See e.g., Lydersen et al., Annals New York Academy of Sciences 745:222-31 (1994).

Various other methods have been developed for isolation and/or purification of antibodies including the application of acid precipitation. See e.g., U.S. Pat. Nos. 7,038,017; 7,064,191; 6,846,410; 5,429,746; 5,151,504; 5,110,913; 4,933,435; 4,841,024; and 4,801,687.

The terms “isolating” and “isolation” refer to separating a biomacromolecule from at least one other undesired component or impurity found in the composition. The term “isolating” includes “purifying” and “clarifying.” No particular level of isolation of a biomacromolecule is required, however in some embodiments, at least 50%, 70%, 80%, 90%, or 95% (w/w) of an impurity is separated from the biomacromolecule. For example, in some embodiments, isolation of a biomacromolecule would comprise separating the biomacromolecule from 80% of the HCP present originally in the composition.

The terms “clarifying” and “clarification” refer to the removal of large particles from a composition. For example, as applied to cellular cultures and growth media, the term “clarifying” refers to, e.g., the removal of prokaryotic and eukaryotic (e.g., mammalian) cells, lipids, and/or nucleic acids (e.g., chromosomal and plasmid DNA) from the cell culture. In some embodiments, the method of the present invention comprises (a) lowering the pH of the composition, allowing an impurity to flocculate within the composition, (b) adding a divalent cation to the composition; and (c) separating the biomacromolecule from an impurity in the composition. No particular level of flocculation of an impurity is required, however in some embodiments, at least 50%, 70%, 80%, 90%, or 95% (w/w) of an impurity is flocculated. For example, in some embodiments, clarification of a biomacromolecule would comprise flocculating 80% of the mammalian cells present in a composition. Flocculation can be measured by methods known to those in the art, including spectrophotographic methods such as a turbidimeter.

The terms “purifying” and “purification” refer to separating the biomacromolecule of the invention from an impurity or other contaminants in the composition, regardless of the size of the impurity. Thus, the term purification would encompass “clarification,” but it would additionally encompass impurities smaller in size than those removed during clarification, e.g., proteins, lipids, nucleic acids, and other forms of cellular debris, viral debris, contaminating bacterial debris, media components, and the like. No particular level of purification of a biomacromolecule is required, however in some embodiments, at least 50%, 70%, 80%, 90%, or 95% (w/w) of an impurity is purified from the biomacromolecule. For example, in some embodiments, purification of a biomacromolecule would comprise separating the biomacromolecule from 80% of the HCP present originally in the composition.

The term “impurity” refers to one or more components of the composition that is different from the biomacromolecule of the present invention. In some embodiments, the impurity can include an intact mammalian cell (e.g., Chinese hamster ovary cells (CHO cells) or murine myeloma cells (NSO cells)), or partial cells, e.g., cellular debris. In some embodiments, the impurity comprises a protein (e.g., soluble or insoluble proteins, or fragments of proteins, such as HCP), lipid (e.g., cell wall material), nucleic acid (e.g., chromosomal or extrachromosomal DNA), ribonucleic acid (t-RNA or mRNA), or combinations thereof, or any other cellular debris that is different from the biomacromolecule of interest. In some embodiments, the impurity can originate from the host organism that produced or contained the biomacromolecule of interest. For example, an impurity could be a cellular component of a prokaryotic or eukaryotic cell (e.g., cell wall, cellular proteins, DNA or RNA, etc.) that expressed a protein of interest. In some embodiments, the impurity is not from the host organism, e.g., an impurity could be from the cell culture media or growth media, a buffer, or a media additive. The impurity as used herein can include a single undesired component, or a combination of several undesired components.

Various means can be used to separate the biomacromolecule of the present invention from one or more impurities. Examples of means of separating the biomacromolecule from an impurity include, without limitation, precipitation, immunoprecipitation, chromatography, filtration, centrifugation, and combinations thereof. In some embodiments, the separating of the biomacromolecule from the impurity is achieved by the use of a filter. The term “filtration” or “filtering” refers to the process of removing suspended particles from a composition by passing the composition through one or more semi-permeable membranes (or medium) of a specified pore size diameter. The term “permeate stream” when referring to filtration, refers to the fraction of the composition that passes through the filter pores during filtration. The term “retentate stream” when referring to filtration, refers to the fraction of the composition that remains on the filter or that does not pass through the filter pores during filtration. In some embodiments, after filtration the biomacromolecule of the present invention is substantially in the permeate stream (i.e., it passes through the filter pores and is collected), while an impurity (e.g., cellular debris, DNA, and/or HCP) is substantially in the retentate stream. In some embodiments, after filtration the biomacromolecule of the present invention is substantially in the retentate stream, while an impurity is substantially in the permeate stream. In some embodiments, “bench scale” filtration can be used to predict appropriate conditions for industrial scale filtration.

Suitable filter types, chemistries, and module configurations for purifying particular biomacromolecules are known to those in the art and can be selected based on various factors, e.g., the amount and size of the components of the composition to be filtered, the volume of the composition to be filtered, and the cell density and viability of the composition to be filtered. In some embodiments, filters, such as membrane filters, plate filters, cartridge filters, bag filters, pressure leaf filters, rotary drum filters or vacuum filters can be used. In some embodiments, a depth filter or a cross filter is used. The types of crossflow filter modules that apply in the present invention include hollow fiber, tubular, flat plate (plate-and-frame), spiral wound, or vortex flow (e.g., rotating) filter geometries. In some embodiments, a tangential flow filter is used. In some embodiments, hollow fibers, tubular, and flat-sheet membrane modules were utilized in a tangential-flow (cross-flow) mode. Commercially available filters that can be employed are manufactured for various manufacturing vendors such as Millipore Corporation (Billerica, Mass.), Pall Corporation (East Hills, N.Y.), GE Healthcare Sciences (Piscataway, N.J.), and Sartorius Corporation (Goettingen, Germany).

The pore diameter in the filters of the present invention can vary according to the type of biomacromolecule being isolated and the type of impurities present in the composition. In some embodiments, the filter pore diameters can be 0.1 μm to 1.0 μm, 0.2 μm to 0.8 μm, or 0.2 μm to 0.65 μm in diameter.

In some embodiments, the present invention is directed to a method of purifying a biomacromolecule in a composition, the method comprising (a) lowering the pH of the composition; (b) adding a divalent cation to the composition; and then (c) filtering the composition through a membrane, the filtering resulting in a transmembrane pressure, wherein the transmembrane pressure remains substantially constant during the filtering. Thus, the term “substantially constant” where referring to the transmembrane pressure, refers to transmembrane pressures that do not increase greater than 4 psi, 3 psi, or 2 psi over the course of filtration.

When isolating biomacromolecules, in some embodiments large volumes of a composition (e.g., harvest feed) can be present, e.g., during commercial manufacturing processes. Large volumes present several challenges for purification processes. For example, the effect that a small change in flow rate through a filter has on the recovery of an isolated biomacromolecule is amplified when large volumes are used. Likewise, when using large volumes, the effect that an increase in cell density in a harvest feed has on product recovery is also amplified. Thus, the use of large volumes of a composition present unique problems that are amplified and have greater ramifications relative to the use of smaller volumes. Thus, in some embodiments the present invention is directed to a method of isolating a biomacromolecule present in a large volume of a composition. The term “large volume” refers to volumes associated with the commercial and/or industrial production of a biomacromolecule. In some embodiments, the term “large volume” refers to 10 to 2000 liters, 20 to 1000 liters or 50 to 500 liters.

### Viral Inactivation

Viral inactivation steps often also occur during BDS manufacturing. Viral inactivation can occur, for example, by use of low pH. Methods of altering pH have been described above and are known to those of skill in the art. Treatment with solvents or detergents, irradiation, brief exposures to high temperatures and viral retentive filters can also be used to accomplish viral inactivation. Methods of viral inactivation are known to those of skill in the art, and one of skill in the art can select a viral inactivation method to be used during BDS manufacturing according to the present invention.

### Drug Product Manufacturing

Biologic product manufacturing steps can be highly dependent on the type of biologic being produced and purified and also on the method in which the biologic will be used. Thus, upstream bulk drug substance manufacturing processes often must be specifically designed to produce a bulk drug substance in a form that is suitable for the necessary downstream drug product manufacturing steps. By decoupling upstream bulk drug substance manufacturing steps from the drug product manufacturing steps through the formation of a stable storage intermediate, the present invention allows for increased efficiency and flexibility in the overall process for manufacturing biologics and also creates an easy to handle intermediate.

In addition, the formation of storage stable intermediates can also be useful between the various steps in the drug product manufacturing process. As is known to those of skill in the art, any number of processes can be used in drug product manufacturing. Such techniques include precipitation, freezing, quick-freezing, sterile filtration (e.g., ultrafiltration or diafiltration), and lyophilization.

For example, ultrafiltration/diafiltration (UF/DF) steps can occur during drug product (DP) manufacturing. Purity of a product after UF/DF (post-UF/DF) is generally at least 99%. Formulation of a stable storage intermediate post-UF/DF results in a high concentration protein formulation for DP presentation and enhances the bulk drug substance (BDS) stability and/or shelf life, even when stored at 2-8 or 25° C. In addition, formation of a stable intermediate at this stage has the advantage of decoupling BDS and DP shelf life and further enhances DP shelf life or the drug delivery mechanism.

### Pharmaceutical Compositions

In some embodiments, the biomacromolecule or composition of the present invention is pharmaceutically acceptable. “Pharmaceutically acceptable” refers to a biomacromolecule or composition that is, within the scope of sound medical judgment, suitable for contact with the tissues of human beings and animals without excessive toxicity or other complications commensurate with a reasonable benefit/risk ratio.

The method of the present invention further provides for administering the final

DP to patients. The route of administration of the DP may be, for example, oral, parenteral, by inhalation or topical. The term parenteral as used herein includes, e.g., intravenous, intraarterial, intraperitoneal, intramuscular, subcutaneous, rectal or vaginal administration. While all these forms of administration are clearly contemplated as being within the scope of the invention, a form for administration would be a solution for injection, in particular for intravenous or intraarterial injection or drip. Usually, a suitable pharmaceutical composition for injection may comprise a buffer (e.g. acetate, phosphate or citrate buffer), a surfactant (e.g. polysorbate), optionally a stabilizer agent (e.g. human albumin), etc. However, in other methods compatible with the teachings herein, the DP of the invention can be delivered directly to the site of the adverse cellular population thereby increasing the exposure of the diseased tissue to the therapeutic agent.

As used herein, the terms “treat” or “treatment” refer to both therapeutic treatment and prophylactic or preventative measures, wherein the object is to prevent or slow down (lessen) an undesired physiological change or disorder, such as the progression of multiple sclerosis. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sports, or pet animals such as dogs, cats, guinea pigs, rabbits, rats, mice, horses, cattle, cows, and so on.

The pharmaceutical compositions containing the DP used in this invention comprise pharmaceutically acceptable carriers, including, e.g., ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Preparations for parenteral administration includes sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In the subject invention, pharmaceutically acceptable carriers include, but are not limited to, 0.01-0.1M and preferably 0.05M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present such as for example, antimicrobials, antioxidants, chelating agents, and inert gases and the like.

More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will preferably be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Suitable formulations for use in the therapeutic methods disclosed herein are described in Remington's Pharmaceutical Sciences, Mack Publishing Co., 16th ed. (1980).

Sterile injectable solutions can be prepared by incorporating an active compound (e.g., a TNFα antibody, or antigen-binding fragment, variant, or derivative thereof, by itself or in combination with other active agents) in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yields a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art. Further, the preparations may be packaged and sold in the form of a kit such as those described in co-pending U.S. Ser. No. 09/259,337 (US 2002/0102208 A1), which is incorporated herein by reference in its entirety. Such articles of manufacture will preferably have labels or package inserts indicating that the associated compositions are useful for treating a subject suffering from, or predisposed to a disease or disorder.

Parenteral formulations may be a single bolus dose, an infusion or a loading bolus dose followed with a maintenance dose. These compositions may be administered at specific fixed or variable intervals, e.g., once a day, or on an “as needed” basis.

Certain pharmaceutical compositions used in this invention may be orally administered in an acceptable dosage form including, e.g., capsules, tablets, aqueous suspensions or solutions. Certain pharmaceutical compositions also may be administered by nasal aerosol or inhalation. Such compositions may be prepared as solutions in saline, employing benzyl alcohol or other suitable preservatives, absorption promoters to enhance bioavailability, and/or other conventional solubilizing or dispersing agents.

With the increase in clinical dosing requirement and different preferred routes of administration (e.g., subcutaneous administration), the need to have high concentration protein formulation is becoming more important. Conventional formulation approaches can be used to formulate a product or protein in liquid or as lyophile. For example, certain subcutaneous formulations have been developed using a histidine formulation at 150 mg/mL of protein concentration. However, such approaches may reach their physical limit once the protein concentration surpasses 150 mg/mL. The limits are attributed to the increase in propensity of protein aggregation and the rise in solution viscosity.

For example, an increase in monoclonal antibody concentration in a formulation has been shown to correlate with a rise in viscosity. Furthermore, increasing concentrations of a monoclonal antibody above 150 mg/mL results in a significant increase in viscosity, which rises at an even faster rate above 200 mg/mL.

The rise in viscosity is related to how well a patient may be able to handle self-administration (syringibility) of a product. Normal hand pressure is generally in the range of 7-10 lbs. A nurse administering a drug is expected to be able to handle about 20-50 lbs. Increasing concentration of a product or protein formulation (resulting in an increased viscosity), requires greater and greater amounts of force to be applied when such a product or protein formulation is administered, for example, by syringe. For example, a monoclonal antibody (mAb) formulation that is 95 mg/mL requires about 40.0 lbs. of force, a mAb formulation of 152 mg/mL requires greater than 40.0 lbs. of force, and a mAb formulation of 205 mg/mL requires over 60 lbs. of force.

### Clinical Manufacturing Control Systems

The most widely adapted standards for manufacturing control systems in the US and Europe are ISA S88.01 and IEC 61512-01 respectively (the disclosures of which are incorporated herein by reference thereto as being widely known in the art). These standards refer to various models such as equipment models and recipe models and the various modules and components involved in manufacturing and batch control. Terminology and methodology used hereinafter are specifically with respect to those defined in such standards and particularly in ISA S88.01 (S88).

Many of the actual processes in batch manufacturing of products such as chemicals, particularly pharmaceuticals and biologicals are run and controlled, in accordance with the S88 standards, using automated computer driven programs. However, the actual design, planning and feedback-quality control have extensive manual components and manual data entries, albeit with the use of computer systems.

Manufacturing plants at pharmaceutical companies and in many other industries are often run on a 24/7 basis and appropriate process design and scheduling of manufacture is an economic necessity but one in which use of conventional computer tools (for example spread sheets) is labor intensive and not well integrated to execution systems. Consequently, the manual entries or calculated results from one production system must be carefully transcribed and constantly verified to ensure that values have not changed at different stages or systems of the process.

Chemical and particularly pharmaceutical production involves the scaling up from laboratory discovery and synthesis to large scale commercial production and batch processes. Batch manufacture of other products and commodities involves analogous scale up and processes. Common steps to achieve this scale-up include the steps of designing a process model (the sequence of steps involved in the manufacturing process) then a plant model (an identification of available equipment at a plant site with capabilities as necessary for effecting the manufacturing steps with correlation thereto) and finally a control model with control parameters and instructions, i.e., operational parameters on the plant model. In this latter stage, recipe configuration data is generated and correlated with electronic work instructions and/or process control systems for material tracking and automated or manual recipe execution. There is an interface to analyze system performance with raw data generation of events, alarms, and user actions all with time stamps. Also collected are process analytical technology (PAT) and conventional instrument data with the generation of reports and process notes as well as the triggering of investigations of events (as needed). As referred to above, production requires scheduling to encompass facilitated manufacture of different products using common equipment as well as to allow factoring in of availability of raw materials and other resources.

In a typical pharmaceutical production timeline in the United States a new product application (NDA) is submitted to the FDA (or equivalent regulatory authority in other countries or regions) together with a production process with basic parameters usually developed in the research lab. The process is then further developed for improvement in terms of yield, purity, economics, raw product availability; etc. Once the process is developed, it is scaled up with equipment needs being defined as well as processing steps and materials involved. Planning and scheduling is then calculated relative to a plant schedule of other product production. Operating instructions are prepared in a pre-campaign set-up and a recipe is formulated for a production execution system which may comprise a DCS (distributed control system), or an Electronic Work Instruction, or other processor, or any combination of these computer based execution systems. A solvent or water run or dry run (if required), or other offline production simulation run is then effected to fine tune the system and the campaign (which defines a sequence of one or more batches) is run. Batches of product (active pharmaceutical product or API) are released, with notation of deviations, changes and review. Deviations are investigated as to source and, with clearance, drug product manufacturing, with the API, begins. Similar design, planning and execution processes are then carried out in drug product manufacturing. In order to maintain quality, efficiency and safety standards and to effect improvements there is a constant monitoring and analysis of all the manufacturing information.

Certain factors should be considered in determining how a the manufacturing of a pharmaceutical formulation should be carried out for a particular product. For example, the type of product itself, i.e., whether the product is a low molecular weight protein, a high molecular weight protein, a peptide, or a small molecule, affects whether that product can be stored, or preferentially stored, in certain storage forms during the manufacturing process. Types of products (e.g., proteins or metabolites) can be classified according to the type of storage intermediate that is suitable for that product at different stages of the manufacturing process. A product formulation classification scheme is as follows, where products have been categorized as Class I, II, III or IV.

TABLE I

| | Class I | Class II | Class III | Class IV |
|---|---|---|---|---|
| BDS | Liquid | Frozen Liquid | Frozen Liquid | Frozen Liquid |
| DP | Liquid | Liquid | Lyophile | Frozen Liquid |

BDS = bulk drug substance
DP = drug product

Class I products include, for example, certain high molecular weight proteins or antibodies, including Tysabri, DEC 152, DEC 114, M200 and Cripto. Class II products include, for example, molecules or enzymes such as PEG-interferon (IFN), Neublastin, IGF1R, AvB6 and GE2. Class III products include, for example, proteins such as LTBR Products of Class IV include, for example, CBE11.

Additional factors should be considered with respect to product formulation. A well-designed manufacturing process is relevant at all stages of product manufacturing, including, but not limited to, the pre-clinical research and development stage, Phase I, II and III clinical trial stages, and the biological license application (BLA) stage. Formulation development cycles overlap the various product manufacturing stages. Cycle 1 starts prior to the pre-clinical research and development stage and can extend through Phase II of the clinical trials. Cycle 2 starts after Phase I, and usually prior to Phase II and extends through the BLA stage.

General assumptions for Cycle 1 of manufacturing are as follows: A bioreactor process involves a 2000L cell culture at 2-3 g/L titer. The BDS generated from a bioreactor process, or per batch, is about 2-3 kg. The BDS is at a 50 mg/mL protein concentration in about 40-60 Liters BDS. Such a supply of BDS may have an extended use beyond Phase I. Thus, a stable intermediate storage form capable of maintaining the BDS in a form that can be subsequently utilized in later product development stages and cycles, is critical to providing flexibility and efficiency of the manufacturing process.

According to the present invention, the process for manufacturing biologics should be compatible with general life cycle manufacturing requirements. To ensure compatibility, a matrix environment or formulation, such as a stable storage intermediate described above ensures handling, storage stability, and shelf life.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., Sambrook et al., ed., Cold Spring Harbor Laboratory Press: (1989); Molecular Cloning: A Laboratory Manual, Sambrook et al., ed., Cold Springs Harbor Laboratory, New York (1992), DNA Cloning, D. N. Glover ed., Volumes I and II (1985); Oligonucleotide Synthesis, M. J. Gait ed., (1984); Mullis et al. U.S. Pat. No: 4,683,195; Nucleic Acid Hybridization, B. D. Hames & S. J. Higgins eds. (1984); Transcription And Translation, B. D. Hames & S. J. Higgins eds. (1984); Culture Of Animal Cells, R. I. Freshney, Alan R. Liss, Inc., (1987); Immobilized Cells And Enzymes, IRL Press, (1986); B. Perbal, A Practical Guide To Molecular Cloning (1984); the treatise, Methods In Enzymology, Academic Press, Inc., N.Y.; Gene Transfer Vectors For Mammalian Cells, J. H. Miller and M. P. Calos eds., Cold Spring Harbor Laboratory (1987); Methods In Enzymology, Vols. 154 and 155 (Wu et al. eds.); Immunochemical Methods In Cell And Molecular Biology, Mayer and Walker, eds., Academic Press, London (1987); Handbook Of Experimental Immunology, Volumes I-IV, D. M. Weir and C. C. Blackwell, eds., (1986); Manipulating the Mouse Embryo, Cold Spring Harbor Laboratory Press, Cold Spring Harbor, N.Y., (1986); and in Ausubel et al., Current Protocols in Molecular Biology, John Wiley and Sons, Baltimore, Md. (1989).

Standard reference works setting forth general principles of immunology include Current Protocols in Immunology, John Wiley & Sons, New York; Klein, J., Immunology: The Science of Self-Nonself Discrimination, John Wiley & Sons, New York (1982); Kennett, R., et al., eds., Monoclonal Antibodies, Hybridoma: A New Dimension in Biological Analyses, Plenum Press, New York (1980); Campbell, A., “Monoclonal Antibody Technology” in Burden, R., et al., eds., Laboratory Techniques in Biochemistry and Molecular Biology, Vol. 13, Elsevere, Amsterdam (1984), Kuby Immunnology 4th ed. Ed. Richard A. Goldsby, Thomas J. Kindt and Barbara A. Osborne, H. Freemand & Co. (2000); Roitt, I., Brostoff, J. and Male D., Immunology 6th ed. London: Mosby (2001); Abbas A., Abul, A. and Lichtman, A., Cellular and Molecular Immunology Ed. 5, Elsevier Health Sciences Division (2005); Kontermann and Dubel, Antibody Engineering, Springer Verlan (2001); Sambrook and Russell, Molecular Cloning: A Laboratory Manual. Cold Spring Harbor Press (2001); Lewin, Genes VIII, Prentice Hall (2003); Harlow and Lane, Antibodies: A Laboratory Manual, Cold Spring Harbor Press (1988); Dieffenbach and Dveksler, PCR Primer Cold Spring Harbor Press (2003).

All of the references cited above, as well as all references cited herein, are incorporated herein by reference in their entireties.

### Examples

### Example 1

Degrees of acceptability regarding different product classes have been assessed (as indicated by the “+” and “−” signs) with respect to stability, drug product (DP) manufacturing, DP presentation, BDS storage, and DP storage. The results presented below show assessment of factors during Cycle 1 of product manufacturing:

TABLE 2
CYCLE 1 FORMULATION

| Formulation | Stability | | | | |
|---|---|---|---|---|---|
| Class | (Shelf | | DP | | DP |
| (BDS/DP) | Life) | DP MFg | Presentation | BDS Storage | storage |
| I (L/L) | + | ++ | + | ++ | + |
| II (FL/L) | ++ | ++ | + | + | + |
| III (FL/Lyo) | ++++ | + | + | + | + |
| IV (FL/FL) | +++ | ++ | − | + | − |

Degrees of acceptability as described above have been assessed for Cycle 2 of product manufacturing as follows:

TABLE 3
CYCLE 2 FORMULATION

| Formulation | Stability | | | | |
|---|---|---|---|---|---|
| Class | (Shelf | | DP | | DP |
| (BDS/DP) | Life) | DP MFg | Presentation | BDS Storage | storage |
| I (L/L) | ++++ | +++ | +++ | +++ | + |
| II (FL/L) | ++++ | +++ | +++ | ++ | ++ |
| III (FL/Lyo) | ++++ | + | + | + | + |
| IV (FL/FL) | +++ | ++ | −−− | + | −−− |

### Example 2

Stable intermediate storage forms are utilized as part of a process for manufacturing a formulation of an antibody or metabolite. The metabolite is produced using a bioreactor process in which cells express the antibody or metabolite. Cells are harvested and then purified using Protein A purification columns. Using water-soluble polymers, the purified protein is co-precipitated into a microsphere, using PROMAXX™ technology to formulate a bulk drug substance (BDS). Alternatively, the protein is crystallized. This BDS is assayed for stability, shelf life and protein concentration.

### Example 3

Storage forms are utilized to formulate the DEC 152 antibody into a drug product (DP) after the production of the antibody is completed. The DEC 152 antibody is produced using a bioreactor process in which cells express the IDEC 152 protein. Cells are harvested and then purified using Protein A purification columns. Purified protein is formulated into a bulk drug substance (BDS). This BDS is fed into the downstream purification process, where the BDS is further purified by ultrafiltration/diafiltration (UF/DF). After UF/DF, water-soluble polymers are utilized to co-precipitate the DEC 152 formulation into a microsphere, using PROMAXX™ technology.

The DEC 152 contained within this microsphere is assayed for stability and protein concentration. In addition, the IDEC 152 is placed in containers, for example, a syringe, and tested for syringibility.

It will be understood by one of ordinary skill in the art that various modifications of the present invention may be made. Accordingly, other embodiments are within the scope of the following claims.

### Example 4

A long term storage intermediate was generated using PEG precipitation and its stability over 135 day period was demonstrated. In these experiments, a 100 ml culture suspension with Chinese Hamster Ovary cells producing a monoclonal antibody was harvested by centrifugation followed by depth filtration. The resulting Harvested Cell Culture Fluid (HCCF) was adjusted to pH 7.2 with 2 M Tris Base at 2-8° C. A single bolus of a stock solution containing 70% (w/w) Polyethylene Glycol (PEG) 3350 and 250 mM Zinc Chloride was added to the adjusted HCCF while vigorously mixing, bringing the sample to a final concentration of 1.5% PEG and 2.5 mM zinc chloride. This material was mixed continuously for 1 hour and the resulting precipitate was centrifuged. The resulting supernatant was decanted, and the resulting pellet was washed with a solution at pH 7.2, containing 1.5% (w/w) PEG and 2.5 mM ZnC1₂ and then mixed. The resulting mixture was again centrifuged and the resulting supernatant was decanted. The resulting precipitate was stored in aseptic conditions and held at room temperature (25° C.), at 2-8° C. or at −20° C. At various time points, a sample containing precipitate stored at each of the three temperatures was resuspended with 100 mM EDTA, 60 mM Acetate, pH 5.0 by rocking. Each sample was analyzed for purity by size exclusion chromatography (SEC), and non-reducing gel-chip electrophoresis. The results of the non-reduced gel chip electrophoresis are shown in Table 2.

TABLE 2
Non-Reduced Gel Chip Electrophoresis Results

Non-Reduced
Gel Chip

| | | | % product | |
|---|---|---|---|---|
| | | | related | % Intact |
| | Time point | | impurity | Antibody |
| | T 0 | | 0.7 | 91.9 |
| −20° C. | 1 | Day | 0.8 | 91.7 |
| | 30 | Days | 0.7 | 92.0 |
| | 75 | Days | 0.9 | 90.5 |
| 2-8° C. | 1 | Day | 0.7 | 91.3 |
| | 30 | Days | 0.7 | 91.3 |
| | 75 | Days | 0.9 | 90.6 |
| 25° C. | 1 | Day | 0.8 | 91.6 |
| | 30 | Days | 0.5 | 83.0 |
| | 75 | Days | <LOQ | 66.9 |

These results indicate that storage at either −20° C. or at 2-8° C. does not alter the composition of the samples, either in terms of the content of intact antibody or in terms of process related impurity, for at least a period of 75 days.

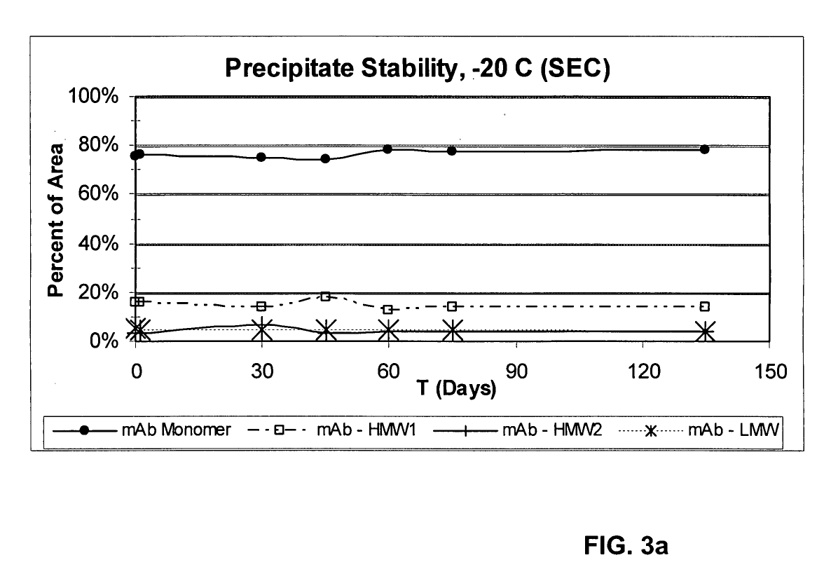

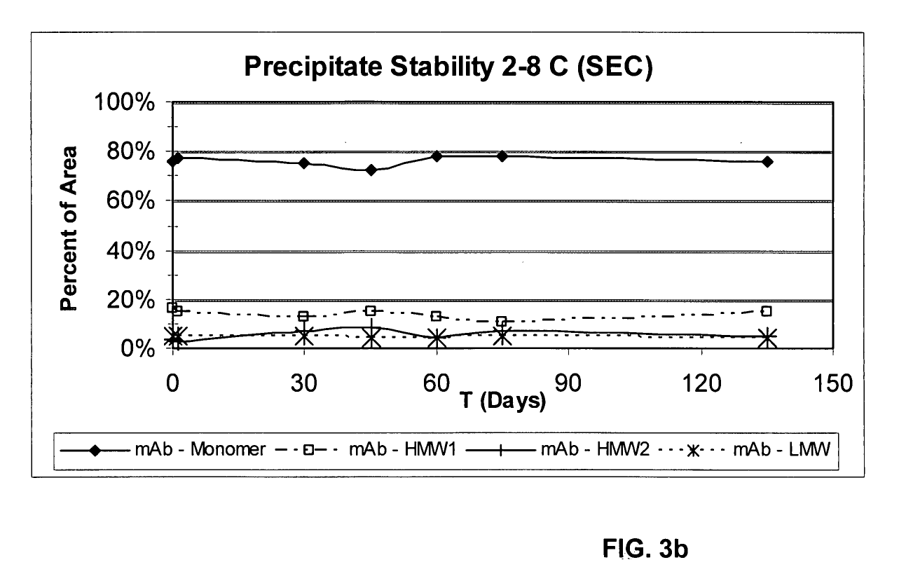

Similarly, size exclusion chromatography results indicate that storage at −20° C. (FIGS. 3a) and 2-8° C. (FIG. 3b) are suitable for storage up to at least 135 days as evidenced by the constant concentration of antibody monomer as well as low (LMW) and high molecular weight (HMW) components.

### Example 5

A long term storage intermediate is generated using PEG precipitation as described above using an industrial-scale manufacturing process. Specifically, a 15,000 liter culture suspension with Chinese Hamster Ovary cells producing a monoclonal antibody is grown in a bioreactor. The suspension is then harvested by centrifugation and filtration. The resulting Harvested Cell Culture Fluid (HCCF) has a volume of approximately 15,000 liters. The HCCF is adjusted to pH 7.2 with 2 M Tris Base at 2-8° C. A single bolus of a stock solution containing 70% (w/w) Polyethylene Glycol (PEG) 3350 and 250 mM Zinc Chloride is added to the adjusted HCCF while vigorously mixing, bringing the sample to a final concentration of 1.5% PEG and 2.5 mM zinc chloride. This material is mixed continuously for 1 hour and the resulting precipitate is centrifuged. The resulting supernatant is decanted, and the resulting pellet is washed with a solution at pH 7.2, containing 1.5% (w/w) PEG and 2.5 mM ZnCl₂ and then mixed. The resulting mixture is again centrifuged and the resulting supernatant is decanted. The resulting precipitate is stored in aseptic conditions and held at room temperature at 2-8° C. or at −20° C. for at least 10 days. The precipitate is then reconstituted, further processed to form a bulk drug substance and finally converted to a drug product.

1. A large-scale process of manufacturing a biologic comprising:
(a) harvesting a biologic from at least about 500 liters of a cell culture;
(b) forming a stable storage intermediate that is capable of being stored for at least 30 days and
(c) storing said intermediate;
(d) further purifying or processing said intermediate and (e) forming a bulk drug substance.

2-15. (canceled)

16. The method of claim 1, wherein said stable storage intermediate is formed by phase separation.

17. The method of claim 16, wherein said stable storage intermediate is formed by precipitation.

18. The method of claim 17, wherein said stable storage intermediate is formed by precipitation with a composition comprising PEG.

19. The method of claim 18, wherein the composition further comprises zinc.

20-39. (canceled)

40. The method of claim 1, wherein said stable storage intermediate is a solid.

41. The method of claim 1, wherein the volume of the cell culture is at least about 1,000 liters.

42. The method of claim 41, wherein the volume of the cell culture is at least about 10,000 liters.

43. The method of claim 1, wherein the cell culture is cultured in a bioreactor, prior to said harvesting step (a).

44. The method of claim 1, wherein said harvesting is achieved by centrifugation.

45. The method of claim 1, wherein said storage is for at least 10 days.

46. The method of claim 45, wherein said storage is for at least three weeks.

47. The method of claim 46, wherein said storage is for at least 75 days.

48. The method of claim 1, wherein said stable storage intermediate is stable at −20° C.

49. The method of any claim 1, wherein said stable storage intermediate is stable at 2-8° C.

50. The method of claim 1, wherein less than 95% of the content of said stable storage intermediate is said biologic.

51. The method of claim 1, wherein said biologic is an antibody.

52. The method of claim 1, wherein said further processing comprises chromatography, filtration, viral inactivation, or lyophilization.

53. The method of claim 1, wherein said method further comprises (f) forming a drug product from said bulk drug substance.

54. The method of claim 53, further comprising (g) administering said drug product to a patient in need thereof.
